(19) United States
(12) Patent Application Publication
(10) Pub. No.: US 2013/0197571 A1
(43) Pub. Date: Aug. 1, 2013

(54) MINIMALLY INVASIVE SURGICAL TECHNIQUES

(71) Applicant: CARDIAPEX LTD., Or Akiva (IL)

(72) Inventors: Ilia HARITON, Zichron-Yaackov (IL); Benad GOLDWASSER, Herzliya Pituach (IL); Boaz MANASH, Givat Ada (IL); Shahar MILLIS, Pardes-Hana-Karkur (IL); Eyal TEICHMAN, Hod-Hasharon (IL)

(73) Assignee: CARDIAPEX LTD., Or Akiva (IL)

(21) Appl. No.: 13/827,744

(22) Filed: Mar. 14, 2013

Related U.S. Application Data
(63) Continuation of application No. 13/818,918, filed on Apr. 24, 2013.

(51) Int. Cl. A61B 17/00

(52) U.S. Cl. CPC A61B 17/0057
USPC 606/213

(57) ABSTRACT

Apparatus and methods are described including a trocar (40) that defines a lumen therethrough, configured to provide a passage through skin of a subject into a body of the subject. A cannula (60) is configured to be placed into the subject's body via the passage provided by the trocar, the cannula being configured to be slidable with respect to the trocar. The cannula includes an outer tube (64) having a first expandable element (77) disposed at a distal end thereof, and an inner tube (62) having a second expandable element (72) disposed at a distal end thereof, the inner tube being configured to be slidable with respect to the outer tube. A vacuum port (61) applied vacuum pressure to the first expandable element via a space (65) between the inner and outer tubes of the cannula. Other applications are also described.

### CROSS REFERENCES TO RELATED APPLICATIONS

The present application claims the benefit of:

U.S. Provisional Patent Application 61/376,897, entitled “Minimally invasive surgical procedure,” filed Aug. 25, 2010;

U.S. Provisional Patent Application 61/452,465, entitled “Minimally invasive surgical techniques,” filed Mar. 14, 2011; and

U.S. Provisional Patent Application 61/475,751, entitled “Minimally invasive surgical techniques,” filed Apr. 15, 2011.

### FIELD OF EMBODIMENTS OF THE INVENTION

Some applications of the invention relate generally to surgical procedures, and more specifically to apparatus and methods for minimally-invasive surgery, such as minimally-invasive cardiac surgery.

### BACKGROUND

Heart valve surgery is used to repair or replace diseased heart valves. Transcatheter alternatives to standard valve implantation, such as aortic valve replacement, have been developed to reduce mortality and morbidity rates in subjects in whom the risk of conventional surgery for valve replacement is considered to be high. Techniques for transcatheter mitral valve replacement are currently being developed.

Transapical transcatheter valve implantation techniques exist and typically involve an incision, for example, a thoracotomy, in order to gain access to the heart.

Transfemoral retrograde valve delivery is also a known procedure for valve replacement; however it is typically limited by the size of the delivery system and is generally not recommended for patients with an existing peripheral vascular disease.

### SUMMARY OF APPLICATIONS OF THE INVENTION

In some applications of the present invention, apparatus and methods for minimally invasive cardiac surgery are provided. For some applications, the apparatus and methods are used to replace and/or repair a defective valve (e.g., an aortic valve, or a mitral valve) or any other cardiac structure. For some applications, the method comprises accessing a subject's cardiac anatomy in a percutaneous manner and delivering a tool into the heart for repair and/or replacement of a cardiac structure. Typically, some methods of the present invention are used in order to perform minimally-invasive implantation or repair of a heart valve. Additionally or alternatively, some methods of the present invention are suitable for use in any other type of cardiac surgery that can be performed with a minimally-invasive approach, such as ablation of a heart wall, implantation of a cardiac assist device, repairing a structural defect of the heart, repair of a failed bioprosthesis, treatment of atrial fibrillation, and/or transvascular approach to repairing or implanting a device in the ascending aorta, the aortic arch, and or the carotid arteries.

In some applications of the present invention, a first catheter is advanced through a peripheral blood vessel of the subject into a chamber of the heart, using known techniques. Once the catheter is in a desired position within the chamber of the heart, a longitudinal element e.g., a guidewire or an additional catheter, is passed through the first catheter, and a passage in a wall of the heart is created from within the chamber of the heart. The longitudinal element is passed through the passage, out of the heart, and through skin of the subject, such that the longitudinal element extends from the heart to the skin. A tool (such as cannula) is then passed into the heart over the longitudinal element. The tool is typically used to facilitate repair and/or replacement of a defective cardiac structure.

There is therefore provided, in accordance with some applications of the present invention, apparatus including:

a trocar that defines a lumen therethrough, configured to provide a passage through skin of a subject into a body of the subject; and

a cannula configured to be placed into the subject's body via the passage provided by the trocar, the cannula configured to be slidable with respect to the trocar, the cannula including:
an outer tube having a first expandable element disposed at a distal end thereof; and
an inner tube having a second expandable element disposed at a distal end thereof, the inner tube being configured to be slidable with respect to the outer tube; and
a vacuum port configured to apply vacuum pressure to the first expandable element via a space between the inner and outer tubes of the cannula.

For some applications, the apparatus further includes a locking mechanism configured to facilitate locking of the inner tube in a fixed position with respect to the outer tube.

For some applications, the first expandable element includes a balloon that is flared in a distal direction.

For some applications, a distal surface of the first expandable element is concave in the distal direction.

For some applications, the second expandable element includes a balloon that is flared in a distal direction.

For some applications, a proximal surface of the second expandable element is convex in a proximal direction.

For some applications, the first expandable element includes a suction cup.

For some applications, the suction cup is configured to assume a curved shape in which a proximal portion of the suction cup is concave in a distal direction, and a distal portion of the suction cup is convex in the distal direction.

For some applications, a distal edge of the suction cup is thickened with respect to other portions of the suction cup.

For some applications, the suction cup is configured to be in a folded configuration during insertion of the suction cup through the trocar, the apparatus including a sheath configured to maintain the suction cup in the folded configuration during the insertion, the suction cup being configured to automatically assume an expanded configuration upon being pushed distally to the sheath.

For some applications, the suction cup includes portions thereof that include a shape-memory alloy, the portions being configured to perform at least one function selected from the group consisting of: causing the suction cup to assume the expanded configuration upon being pushed distally to the sheath, and preventing the suction cup from folding upon being pushed against a surface of the subject's body.

For some applications, the distal end of the inner tube is configured to be placed inside a heart of the subject, and provide a working channel from outside the subject's skin to inside the subject's heart.

For some applications, the distal end of the inner tube is configured to be inserted into the subject's heart via a passage in the subject's heart, the first expandable element is configured to seal the cannula with respect to an outer surface of the heart at the passage, and the second expandable element is configured to seal the cannula with respect to an inner surface of the heart at the passage.

There is further provided, in accordance with some applications of the present invention, apparatus for use with an insertion device, the apparatus including a closure device, the closure device including:

a plug portion configured to be placed within a passage in a wall of a heart of a subject by being introduced to the passage via the insertion device, the insertion device being configured to maintain the plug portion in a constrained state thereof during the insertion, the plug portion being configured to automatically increase a radius of the plug portion by more than 0.5 percent by assuming a non-constrained state thereof by being pushed out of a distal end of the insertion device;

an intracardiac portion, coupled to the plug portion, and configured for placement within a heart chamber; and

an extracardiac portion coupled to the plug portion and configured for placement outside of the heart chamber.

For some applications, the plug portion includes a soft outer layer thereof.

For some applications, a radius of the passage is defined by an outer radius of the insertion device, while the plug portion is in the constrained state thereof, the radius of the plug portion is less than the outer radius of the insertion device, and upon assuming the non-constrained state thereof, the radius of the plug portion is at least equal to the outer radius of the insertion device.

For some applications, upon assuming the non-constrained state thereof, the plug portion is configured to seal the passage by expanding to occupy the passage.

For some applications, the plug portion is configured to automatically increase the radius of the plug portion by more than 5 percent upon assuming the non-constrained state thereof, by being pushed out of a distal end of the insertion device.

For some applications, the plug portion is configured to automatically increase the radius of the plug portion by less than 100 percent upon assuming the non-constrained state thereof, by being pushed out of a distal end of the insertion device.

For some applications, the plug portion is configured to increase the radius of the plug portion even in an absence of any radial expansion of the plug portion that is due to absorbance of fluid by the plug portion.

For some applications, the plug portion is configured to further expand upon being placed inside the passage by absorbing fluid while the plug portion is within the passage.

For some applications, the extracardiac portion is shaped to define a disc that is convex in a distal direction.

For some applications, the plug portion is configured, in the absence of any force applied to the plug portion, to reduce a length of the plug portion by 0.5-50 percent.

For some applications, the apparatus further includes an element, configured to draw the intracardiac portion and the extracardiac portion closer to each other.

For some applications, the plug portion is bioabsorbable or biodegradable.

For some applications, more than 50 percent of a non-constrained volume of the plug portion includes an expansible material.

For some applications, the plug portion is configured to facilitate insertion of a device therethrough, into the subject's heart, by the device being advanced through a hole in the plug portion, and the plug portion is configured to automatically seal the passage in the heart subsequent to removal of the device, by the plug portion expanding to seal the hole in the plug portion.

For some applications, the intracardiac portion is shaped to define a disc that is concave in a distal direction.

For some applications, the extracardiac portion is shaped to define a disc that is concave in the distal direction.

For some applications, a radius of curvature of the extracardiac portion is less than a radius of curvature of the intracardiac portion.

For some applications, the intracardiac portion, the extracardiac portion and the plug portion are moveable with respect to each other.

For some applications, the closure device is configured to conform with anatomical variations of the subject's heart, by the intracardiac portion, the extracardiac portion and the plug portion being moveable with respect to each other.

For some applications, the closure device is configured to seal the passage in the wall of the heart by being placed inside the passage, and the closure device is configured to maintain the seal of the passage, by the intracardiac portion, the extracardiac portion and the plug portion being moveable with respect to each other.

For some applications, the intracardiac portion and the extracardiac portion of the closure device are configured to be maintained in folded configurations thereof during insertion of the closure device via the insertion device, and the intracardiac portion and the extracardiac portion are configured to automatically assume unfolded states thereof by being pushed out of the distal end of the insertion device.

For some applications, the intracardiac portion and the extracardiac portion include a shape memory material that is configured to cause the intracardiac portion and the extracardiac portion to automatically assume the unfolded states.

For some applications, the plug portion is configured to increase the radius of the plug portion upon absorbing body fluid.

For some applications, the plug portion is configured to reduce the length of the plug portion by 0.5-50 percent, upon absorbing the body fluid.

There is additionally provided, in accordance with some applications of the present invention, apparatus for use with an insertion device, the apparatus including a closure device, the closure device including:

a support element configured to be placed within a passage in a wall of a heart of a subject by being introduced to the passage while coupled to the insertion device, and to become decoupled from the insertion device subsequent to placement of the support element within the passage;

an inflatable intracardiac portion, coupled to the support element, and configured to be inflated within a heart chamber; and

an inflatable extracardiac portion coupled to the support element, and configured to be inflated outside of the heart chamber.

For some applications, the support element defines a lumen therethrough and the closure device includes at least one hemostatic valve disposed within the lumen.

For some applications, the support element defines a lumen therethrough and the closure device includes a plug configured to be placed within the lumen such as to seal the lumen.

For some applications, the apparatus further includes a thermosetting material that changes from a fluid state to a solid state thereof, the inflatable intracardiac portion is configured to be inflated with the material while the material is in the fluid state thereof, and the material is configured to change to the solid state thereof while the material is within the intracardiac portion.

For some applications, the apparatus further includes a material that changes from a fluid state to a solid state thereof, the inflatable extracardiac portion is configured to be inflated with the material while the material is in the fluid state thereof, and the material is configured to change to the solid state thereof while the material is within the extracardiac portion.

There is further provided, in accordance with some applications of the present invention, apparatus including a closure device, the closure device including:

a plug portion configured for placement within a passage in a wall of a heart of a subject;

an intracardiac portion, coupled to the plug portion, and configured for placement within a heart chamber, and having a radius of curvature; and

an extracardiac portion coupled to the plug portion and configured for placement outside of the heart chamber, having a radius of curvature that is less than the radius of curvature of the intracardiac portion.

There is additionally provided, in accordance with some applications of the present invention, apparatus including a kit including:

a longitudinal element, configured to extend through a peripheral blood vessel of a subject, to transvascularly reach a heart of the subject, and to transmurally pass out of a passage in the heart of the subject and reach skin of the subject via a path extending from the heart to the skin, the longitudinal element including:
a first, soft, distal portion; and
a second, stiffer, proximal portion, coupled or couplable to the first portion.

For some applications, the first and second portions are coupled to each other in the kit.

For some applications, the kit further includes a connection element, which couples a proximal end of the distal portion to a distal end of the proximal portion.

For some applications, the connection element includes a crimping tube.

For some applications, the connection element includes a friction-based connection element.

There is further provided, in accordance with some applications of the present invention, apparatus including:

a catheter including a proximal portion and a distal portion, the catheter being advanceable through a peripheral blood vessel into a left ventricle of a heart of a subject; and

an expandable structure coupled to the distal end of the catheter and configured to expand such that the expandable structure assumes an expanded state thereof within the left ventricle in a vicinity of an apex of the left ventricle, such that a distal end of the expandable structure protrudes distally from the distal end of the catheter.

For some applications, the expandable structure is configured to expand within the left ventricle such that the distal end of the catheter is maintained at a distance from the apex of the left ventricle.

For some applications, the expandable structure is shaped to define a mesh.

For some applications, the expandable structure includes a balloon.

For some applications, the balloon defines bulges on an outer surface thereof, the bulges being configured to generate friction between the outer surface of the balloon and an inner wall of the heart at the apex.

For some applications, the expandable structure includes a metal.

For some applications, the expandable structure includes nitinol.

For some applications, the apparatus further includes a puncturing tool, passable through the catheter, and configured to puncture the apex.

For some applications, the expandable structure is configured to seal the puncture in the apex.

For some applications, the distal end of the catheter is steerable, and the expandable structure provides a space in which the distal end of the catheter can steer while not contacting the apex.

For some applications, the apparatus further includes a puncturing tool, passable through the catheter, and configured to extend from within the space provided by the expandable structure and puncture the apex.

For some applications, the expandable structure is configured to seal the puncture in the apex.

There is additionally provided, in accordance with some applications of the present invention, apparatus including:

a catheter including a proximal portion and a distal portion, the distal portion including:
a curved portion which is configured to conform to an anatomical structure of a body lumen; and
an aperture portion, shaped to define one or more apertures in a lateral surface of the catheter, which are configured to allow passage of a longitudinal element therethrough.

There is further provided, in accordance with some applications of the present invention, apparatus including a kit, the kit including:

at least one hollow surgical needle that is flexible in one region thereof and less flexible at another region thereof.

For some applications, the region that is flexible is shaped to define one or more slits therein, which facilitate the flexibility of the region.

For some applications, the less flexible region of the hollow surgical needle is substantially inflexible.

For some applications, the kit includes a suture, passable through the hollow surgical needle.

For some applications, the at least one hollow surgical needle includes 2-8 hollow surgical needles.

For some applications, the 2-8 hollow surgical needles include 3-5 hollow surgical needles.

For some applications, the needle is configurable to have a J-shape.

For some applications, a sharp distal tip of the J-shaped needle points in a direction that is parallel to a straight portion of the J-shaped needle.

For some applications, a smallest radius of curvature along the J-shaped needle is 1-8 mm.

For some applications, a length of a post-curve distal region of the J-shaped needle is 3-200 mm.

For some applications, a length of a post-curve distal region of the J-shaped needle is 10-20 mm.

For some applications, a distance between a straight portion of the needle and a distal sharp tip of the needle is 2-15 mm.

There is further provided, in accordance with some applications of the present invention, a method including:

advancing a longitudinal element, through a peripheral blood vessel, to a chamber of a heart of a subject;

creating a passage in a wall of the heart; and

passing the longitudinal element through the passage, out of the heart, and through skin of the subject, such that the longitudinal element extends in a path from the heart to the skin.

For some applications, the method further includes advancing a catheter through the blood vessel, and advancing the longitudinal element includes advancing the longitudinal element through the catheter after the catheter has been advanced through the blood vessel.

For some applications, the longitudinal element includes a guidewire, and advancing the longitudinal element includes advancing the guidewire.

For some applications, passing the longitudinal element through the skin includes pulling the longitudinal element through the skin.

For some applications, passing the longitudinal element through the skin includes pushing the longitudinal element through the skin.

For some applications, the method further includes passing a tool over the longitudinal element, toward the heart, on the path extending from the heart to the skin.

For some applications, the method further includes passing a tool over the longitudinal element, from the peripheral blood vessel, to the chamber of the heart.

For some applications, the method further includes passing an additional tool over the longitudinal element, toward the heart, on the path extending from the heart to the skin.

For some applications, the method further includes utilizing the additional tool in conjunction with the tool passed from the peripheral blood vessel to the chamber of the heart.

For some applications, the method further includes coupling the additional tool to the tool passed from the peripheral blood vessel to the chamber of the heart.

There is further provided, in accordance with some applications of the present invention, a method including:

advancing a catheter, through a peripheral blood vessel, to a chamber of a heart of a subject;

passing a longitudinal element through the catheter;

creating a passage in a wall of the heart, from within the chamber of the heart;

passing the longitudinal element through the passage, out of the heart, and through skin of the subject, such that the longitudinal element extends from the heart to the skin; and

subsequently, passing a tool into the heart over the longitudinal element.

For some applications, the method further includes passing a dilator from the skin over the longitudinal element, and enlarging a path to the heart using the dilator.

For some applications, creating the passage in the wall of the heart includes puncturing the wall of the heart with a needle.

For some applications, advancing the catheter includes advancing the catheter over an angiographic guidewire.

For some applications, passing the longitudinal element through the passage out of the heart, includes advancing the longitudinal element through the passage out of the heart.

For some applications, passing the longitudinal element through the passage out of the heart, includes pulling the longitudinal element through the passage out of the heart.

For some applications, the longitudinal element includes a guidewire and the method includes passing the guidewire through the catheter.

For some applications, the longitudinal element includes a second catheter and the method further includes passing the second catheter through the catheter.

For some applications, the tool includes a cannula, and passing the tool into the heart includes passing the cannula into the heart over the longitudinal element.

For some applications, passing the cannula into the heart over the longitudinal element includes:

placing an outer tube of the cannula against on outer surface of the heart, the outer tube having a first expandable element disposed at a distal end thereof; and

placing an inner tube of the cannula inside the heart, the inner tube having a second expandable element disposed at a distal end thereof, the inner tube being configured to be slidable with respect to the outer tube.

For some applications, the method further includes generating a vacuum between the first expandable element and an outer surface of the wall of the heart by applying vacuum pressure via a space between the inner and outer tubes of the cannula.

For some applications, passing the cannula into the heart over the longitudinal element includes:

placing a trocar between ribs of the subject, the trocar defining a lumen therethrough, and

inserting the cannula through the lumen defined by the trocar.

For some applications, the method further includes sealing an inner surface of the wall of the heart by sliding the inner tube of the cannula proximally with respect to the outer tube, such that the second expandable element is placed in contact with the inner surface of the wall of the heart.

For some applications, the method further includes locking a position of the inner tube with respect to the outer tube, subsequent to the placement of the second expandable element in contact with the inner surface of the wall of the heart.

For some applications, a balloon is coupled to a distal end of the cannula, and the method further includes:

inflating the balloon while the balloon is in the heart; and

pulling a proximal end of the cannula in a direction that is away from the body of the subject, such that the heart is pulled towards a chest wall of the subject.

For some applications, the method further includes advancing a prosthetic valve through the cannula.

For some applications, advancing the catheter through a peripheral blood vessel includes advancing the catheter through an artery.

For some applications, advancing the catheter includes advancing the catheter through a femoral artery.

For some applications, advancing the catheter includes advancing the catheter through a radial artery.

For some applications, advancing the catheter through a peripheral blood vessel includes advancing the catheter through a vein.

For some applications, advancing the catheter includes advancing the catheter through a femoral vein.

For some applications, advancing the catheter includes advancing the catheter through a radial vein.

For some applications, creating the passage in the wall of the heart includes puncturing the wall of the heart with the longitudinal element.

For some applications, passing the longitudinal element through the catheter includes passing a longitudinal element having a pointed tip through the catheter.

There is additionally provided, in accordance with some applications of the present invention, a method including:

advancing a catheter into a body lumen of a subject;

passing through the catheter, a guidewire having proximal and distal ends thereof, the distal end having a straight configuration while being passed through the catheter;

creating a passage in a wall of the body lumen from within the lumen;

advancing the guidewire through the passage, out of the lumen, such that the distal end of the guidewire assumes a curved configuration upon exiting the lumen;

passing the guidewire through skin of the subject, such that the guidewire extends from the wall of the body lumen to the skin; and

subsequently, passing a tool into the body lumen over the guidewire.

For some applications, the body lumen includes a heart chamber of the subject, and advancing the catheter into the body lumen includes advancing the catheter into the heart chamber.

For some applications, the method further includes rotating the distal end of the guidewire while the guidewire is outside the heart chamber.

For some applications, the method further includes operating a magnet to rotate the distal end of guidewire while the guidewire is outside the heart chamber.

There is further provided, in accordance with some applications of the present invention, a method including:

advancing a guidewire into a chamber of a heart of a subject;

creating a passage in a wall of the heart, from within the chamber of the heart;

passing the guidewire through the passage, out of the heart, and through skin of the subject, such that the guidewire extends from the heart to the skin;

subsequently, passing a closure device into the heart over the guidewire to seal the passage in the wall of the heart, the closure device including:
a plug portion configured for placement within the passage;
an intracardiac portion, coupled to the plug portion, and configured for placement within the heart chamber;
an extracardiac portion coupled to the plug portion and configured for placement outside the heart chamber; and

inserting the closure device through the passage such that the intracardiac portion and the extracardiac portions apply a force to the plug portion to maintain the plug portion in place within the passage.

For some applications, the plug portion includes an intramural plug portion and passing a closure device into the heart includes passing the intramural plug portion into the heart.

For some applications, inserting the closure device through the passage includes introducing the insertion device to the passage via an insertion device, the insertion device being configured to maintain the plug portion in a constrained state thereof during the insertion, the plug portion being configured to automatically increase a radius of the plug portion by more than 0.5 percent by assuming a non-constrained state thereof by being pushed out of a distal end of the insertion device

There is additionally provided, in accordance with some applications of the present invention, a method including:

placing a guidewire between a body lumen and skin of a subject;

subsequently, passing over the guidewire towards the body lumen an inner tube having distal and proximal ends thereof, the distal end configured to penetrate the passage in the wall of the body lumen;

subsequently, passing over the inner tube towards the body lumen, an outer tube surrounding the inner tube, having distal and proximal ends thereof, the distal end configured to contact an external side of the wall of the body lumen in a vicinity of the passage;

providing at least one suture having proximal, distal and tissue-engaging portions;

advancing the distal portion of the suture through the inner tube and penetrating tissue adjacent to the passage with the tissue-engaging portion; and

subsequently, passing the distal portion of the suture through the outer tube towards the skin of the subject.

For some applications, the method further includes manipulating the proximal and distal portions of the suture from outside the body.

For some applications, manipulating the proximal and distal portions of the suture includes tying the proximal and distal ends of the suture into a knot.

The present invention will be more fully understood from the following detailed description of applications thereof, taken together with the drawings, in which:

### BRIEF DESCRIPTION OF THE DRAWINGS

FIGS. 1A-F are schematic illustrations of respective steps of a minimally invasive surgical procedure performed in accordance with some applications of the present invention;

FIG. 2 is a flowchart describing steps of a minimally invasive surgical procedure performed in accordance with some applications of the present invention;

FIGS. 3A-H are schematic illustrations of a femorally-inserted catheter or a radially-inserted catheter located in a left ventricle of a subject, and of a protective, fixation, and/or locating structure located against the apex of the left ventricle, in accordance with some applications of the present invention;

FIGS. 4A-C are schematic illustrations of devices for receiving and directing a guidewire upon exiting the apex, in accordance with some applications of the present invention;

FIGS. 5A-B are schematic illustrations of a trocar configured to be placed between a subject's ribs, in accordance with some applications of the present invention;

FIGS. 6A-D are schematic illustrations of a cannula for inserting through the trocar and through a hole in the apex of the subject's heart, in accordance with some applications of the present invention;

FIG. 7 is a schematic illustration of a cannula having a plurality of balloons disposed thereon, in accordance with sone applications of the present invention;

FIGS. 8A-B are schematic illustrations of a closure device for sealing a passage in the heart, disposed on a cannula, in accordance with some applications of the present invention;

FIGS. 9A-F are schematic illustrations of closure devices for sealing the passage in the heart, in accordance with some applications of the present invention;

FIGS. 10A-D are schematic illustrations of a closure device being placed within a passage in the heart such as to close the passage, in accordance with some applications of the present invention; and

FIGS. 11A-D are schematic illustrations of a suturing system for sealing a passage in the heart, in accordance with some applications of the present invention.

### DETAILED DESCRIPTION OF EMBODIMENTS

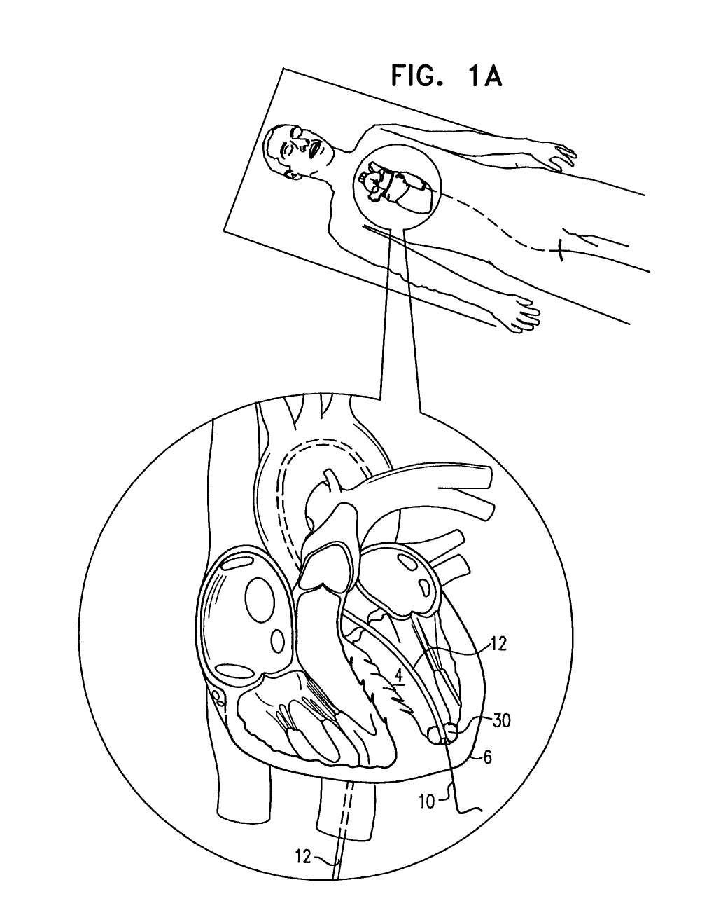

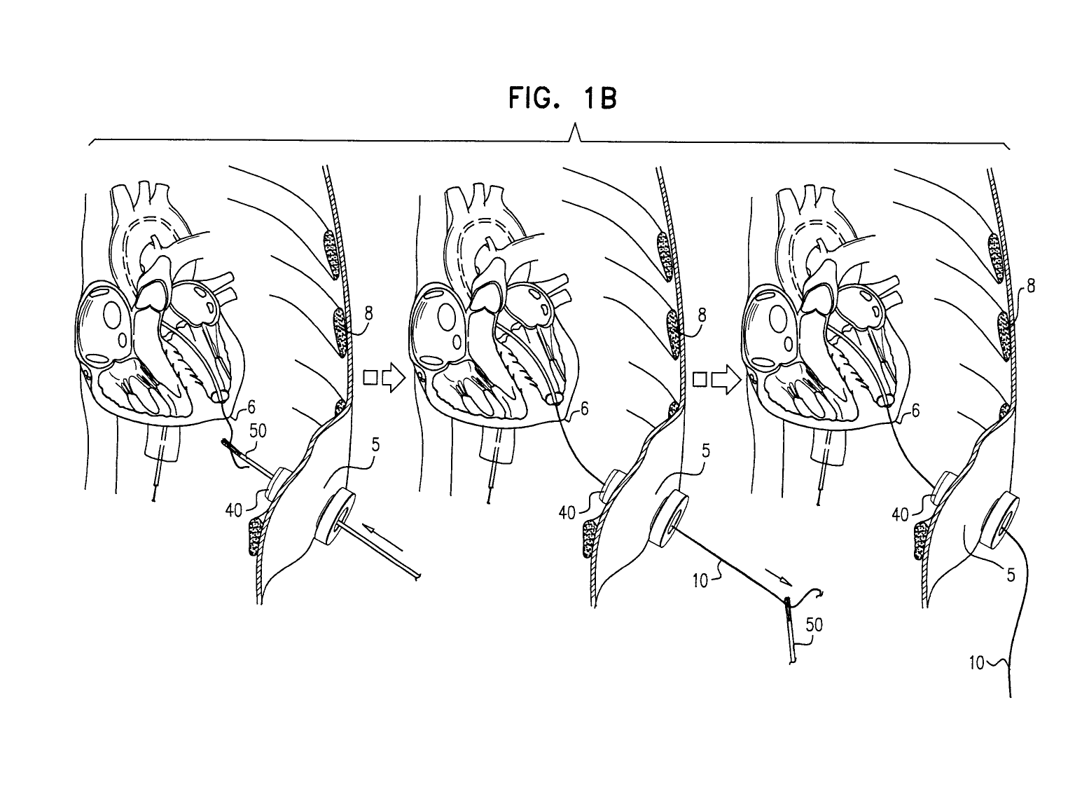

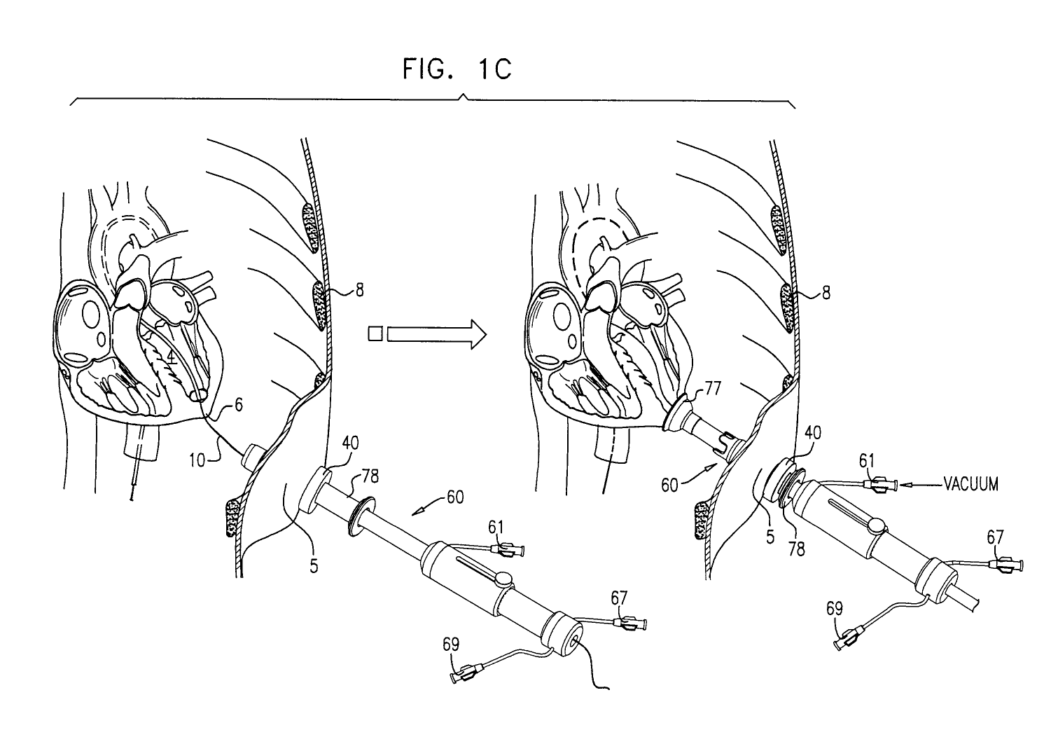

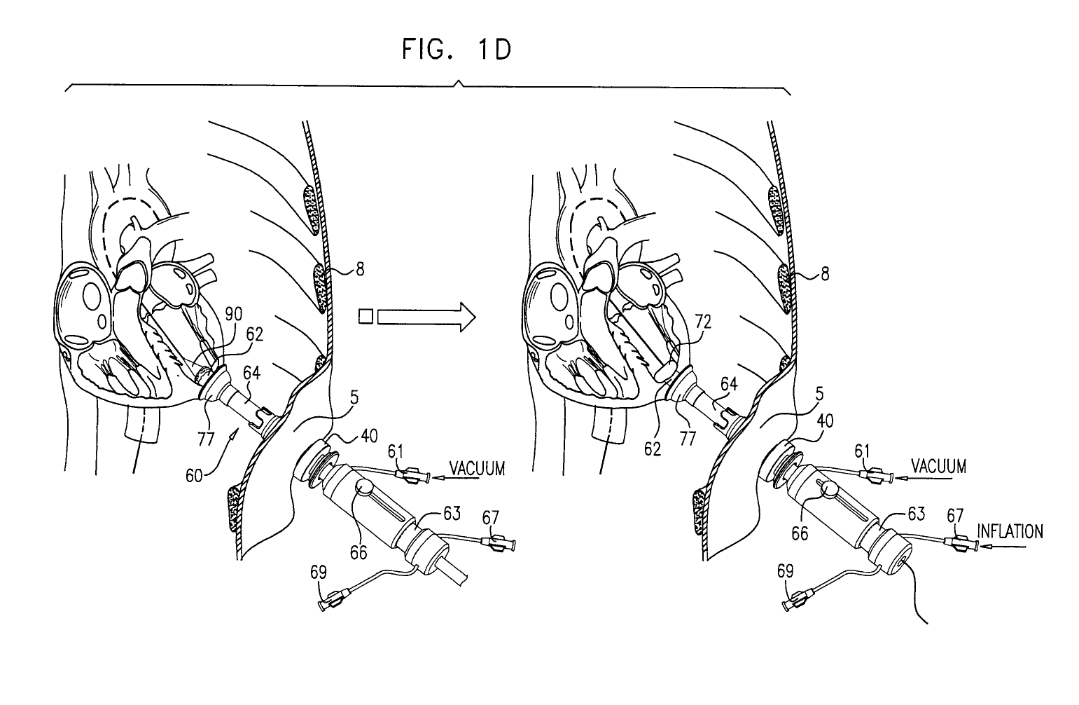

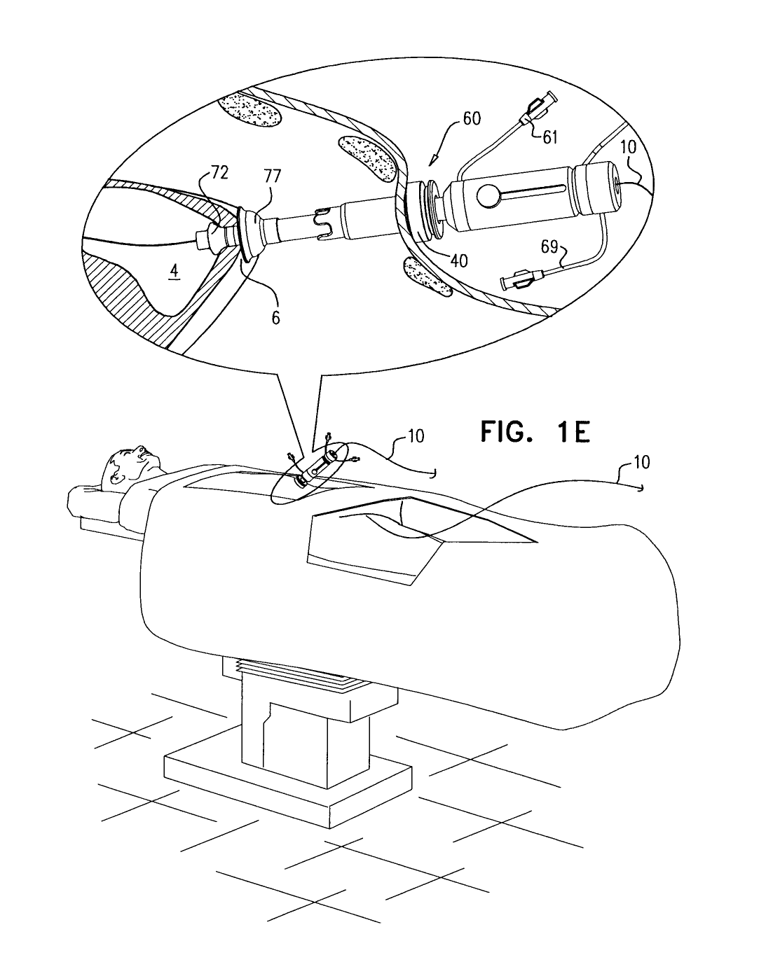

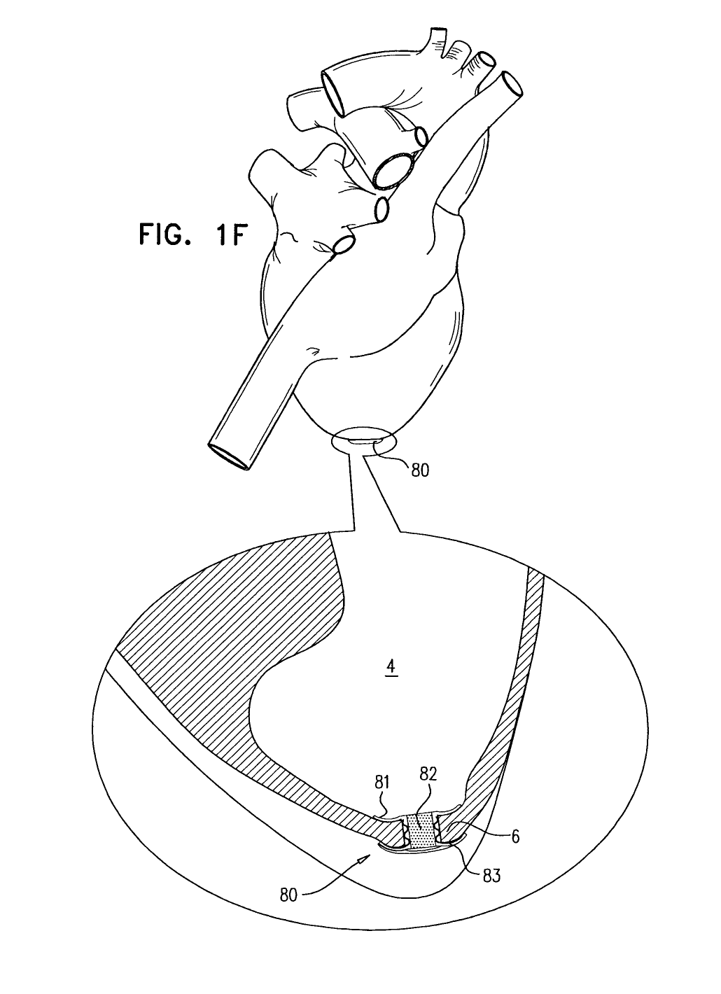

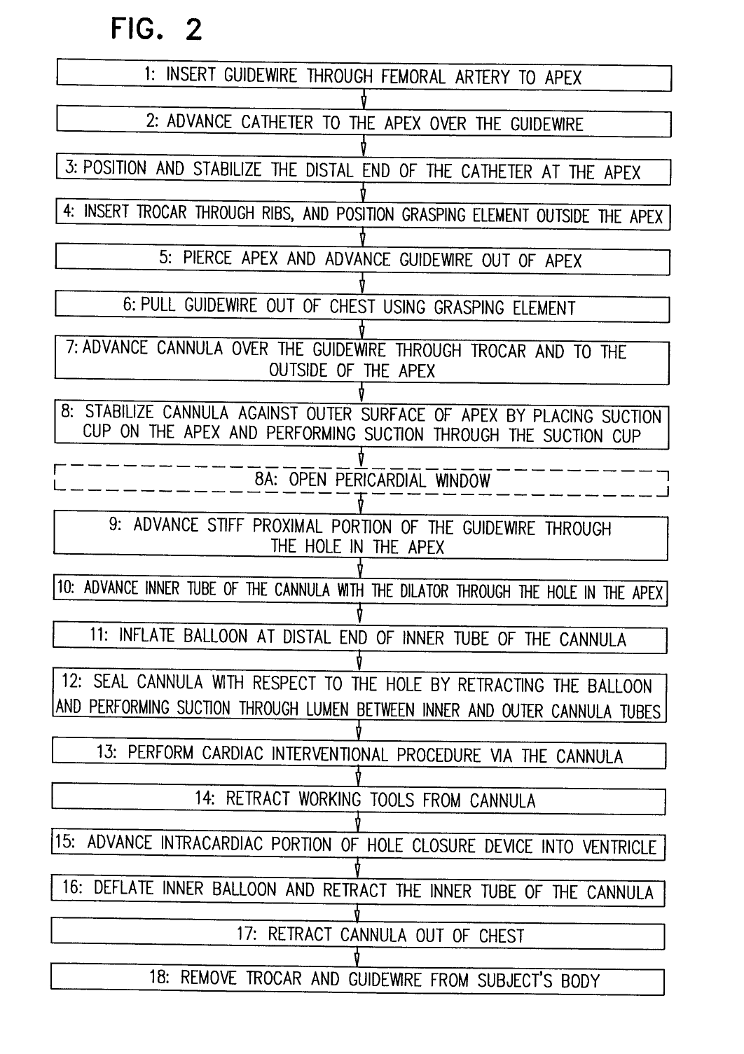

Reference is made to FIGS. 1A-F and FIG. 2, which are schematic illustrations of steps of a minimally invasive surgical procedure and a flowchart describing the steps, in accordance with some applications of the present invention. More specifically, FIGS. 1A-F show, by way of illustration and not limitation, procedures comprising safely positioning a catheter in the left ventricle of the heart and creating a passage in the apex of the left ventricle, such that a guidewire is passed through the catheter, and through the passage in the heart. It is noted that, for some applications, some of the steps of the procedure of the flowchart of FIG. 2 are optional, and that the scope of the present invention includes performing some of the steps of the procedure of the flowchart of FIG. 2, without necessarily performing all of the steps of the procedure.

In Step 1 of the procedure, a guidewire 10 is inserted through a peripheral blood vessel (e.g., the radial artery or the femoral artery, as shown) to apex 6 of the subject's left ventricle 4. For example, the guidewire may be a 0.089 cm (0.035 inch) soft and flexible guidewire. For some applications, the guidewire is an angiographic guidewire. As described hereinbelow, for some applications, the stiffness of the guidewire varies along the length of the guidewire.

In Step 2 of the procedure, when a distal portion of the guidewire is positioned at the apex, a catheter 12 is advanced over the guidewire. It is noted that catheter 12 is shown as a femoral catheter by way of illustration and not limitation. Catheter 12, as described herein in the specification and in the claims may be advanced to the heart through any suitable blood vessel, for example, through the radial artery. Catheter 12 is advanced over the guidewire to a desired location within the chamber of the heart. For some applications, catheter 12 comprises a 4-14 Fr catheter, e.g., a 4-9 Fr catheter. (The units of “Fr” are defined as Diameter (mm)=Fr/3, thus 9 Fr=3 mm.) Typically, the catheter is advanced over the guidewire through the femoral artery, toward the heart, in a retrograde direction, up the aorta and across the aortic valve into the left ventricle. Depending on the state of the native aortic valve, the surgeon may dilate the valve, prior to advancing the catheter into the left ventricle. For some applications, the catheter is inserted through the femoral vein, into the right atrium. The catheter is then passed from the right atrium through the left atrium via the interatrial septum, and then into the left ventricle via the mitral valve, in accordance with techniques that are known in the art.

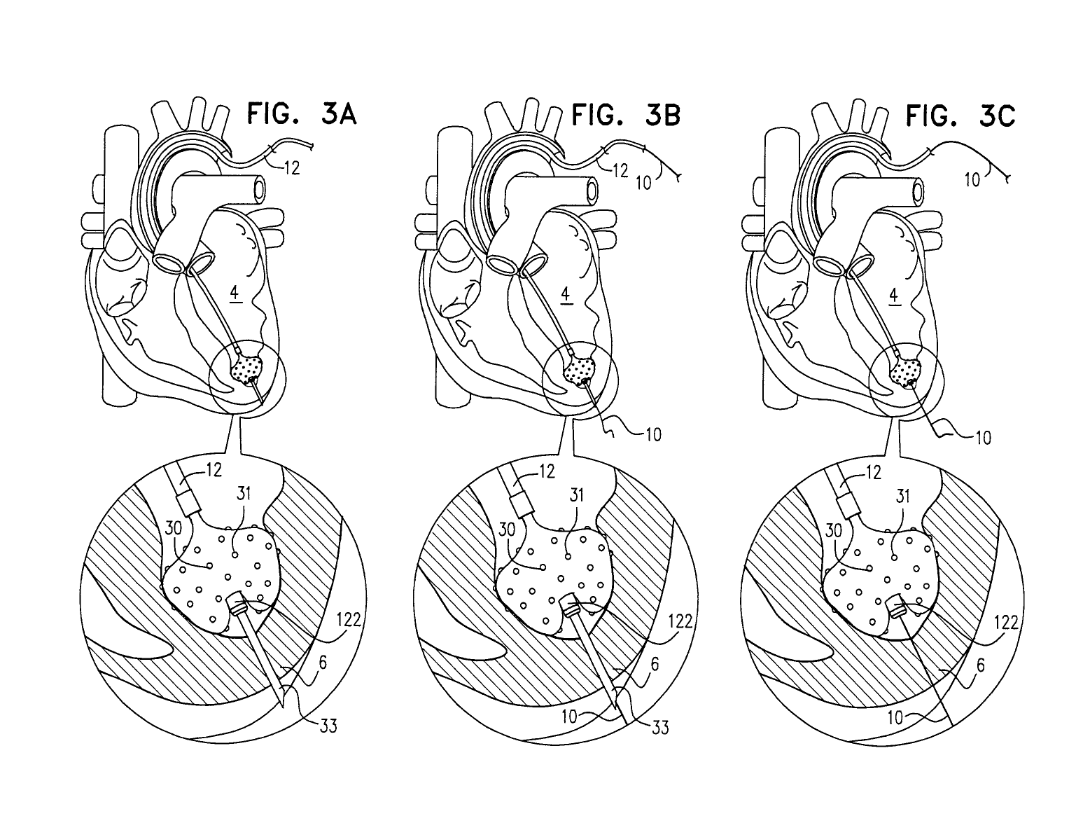

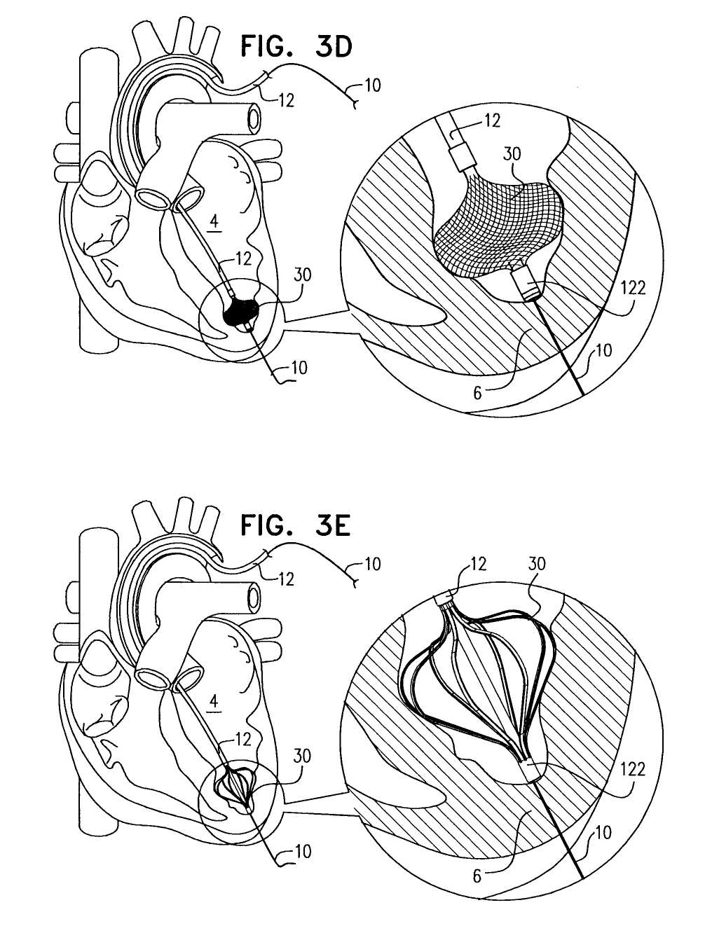

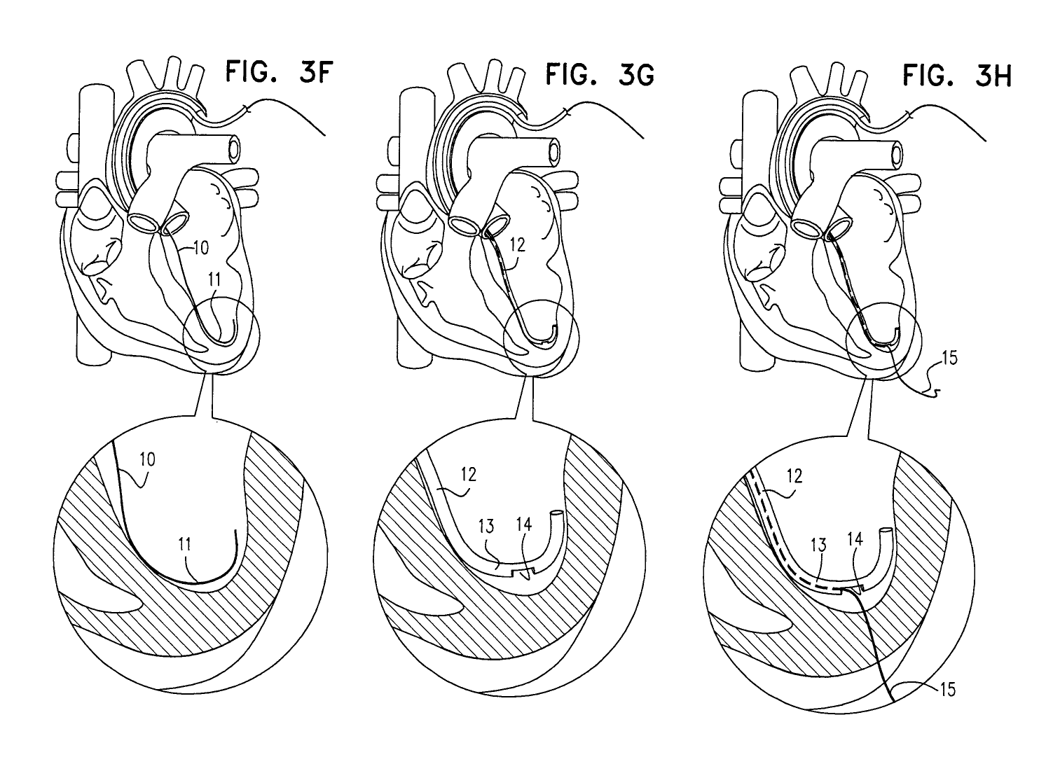

In Step 3 of the procedure, a protective, fixation, and/or locating structure 30 at the distal end of catheter 12 positions and/or fixates the distal end of the catheter at apex 6 of left ventricle 4. Protective, fixation, and/or locating structure 30 is described in more detail hereinbelow with reference to FIGS. 3A-F. For some applications, the distal end of the catheter is placed at the apex in the absence of structure 30.

In Step 4 of the procedure, a trocar 40 (FIG. 1B) is inserted through chest wall 5 between two of the subject's ribs 8, e.g., between the fourth and fifth, the fifth and the sixth, and/or the sixth and the seventh ribs, and a grasping element 50 (FIG. 1B) is inserted into the subject's chest cavity via trocar 40. The grasping element is positioned outside the apex of the subject's heart. Typically, the trocar is inserted between the fifth and the sixth ribs. It is noted that Step 4 is not necessarily performed subsequently to Step 1-3. Rather, Step 4 may be performed before, or simultaneously with, any of Steps 1-3.

In Step 5 of the procedure, subsequent to the distal end of the catheter being positioned and/or fixated at apex 6 of left ventricle 4, a hole is pierced through the apex. For example, an inner catheter having a sharp tip (e.g., catheter 33, shown in FIGS. 3A-B) may be pushed out of the distal end of catheter 12, such as to pierce the apex. Guidewire 10 is advanced through the hole in the apex. For example, the inner catheter, which is used to pierce the hole, may define a lumen therethrough, and the guidewire may be inserted through the hole in the apex via the lumen defined by the inner catheter. Typically, the inner catheter is withdrawn into catheter 12, subsequent to the guidewire having exited the hole in the apex.

FIG. 1A shows the subject subsequent to step 5 of the procedure. As shown, the distal end of catheter 12 is located in a vicinity of apex 6. Structure 30 fixates the distal end of the catheter at the vicinity of the apex. As described in further detail hereinbelow, structure 30 typically protects the apex from being damaged by the distal end of catheter 12, and/or by the inner catheter that is used to pierce the hole in the apex, in addition to stabilizing the distal end of the catheter at the apex. Alternatively or additionally, structure 30 seals the hole that is pierced through the apex. Guidewire 10 is shown in FIG. 1A as having been inserted through the hole in the apex of the subject's ventricle, such that the distal end of the guidewire is disposed outside the subject's heart within the subject's chest cavity. Thus, the distal end of the guidewire passes into the heart via a peripheral artery, and then passes from inside the heart to outside the heart through a hole that has been pierced through the myocardium. It is noted that, in the context of the present application, the term myocardium is used to denote the endocardium, the myocardium and the epicardium, except where stated otherwise, explicitly or implicitly.

In Step 6 of the procedure, grasping element 50 is used to grasp the distal end of guidewire 10, which is disposed inside the chest cavity. The guidewire is then advanced through the passage in the apex by feeding the proximal end of the guidewire through the femoral access point, and, as the distal end of the guidewire advances into the subject's chest cavity, the distal end of the guidewire is pulled to outside the subject's chest, via the trocar. Step 6 is shown in FIG. 1B.

It is noted that, as an alternative to performing Steps 4-6 (i.e., inserting trocar 40 through the subject's ribs, and grasping the distal end of the guidewire with grasping element 50), for some applications, the distal end of the guidewire is guided through the chest cavity, to a location between the subject's ribs. A hole is pierced in the subject's chest wall, between the subject's ribs, from inside the subject's chest cavity (e.g., using an inner catheter having a pointed tip, such as catheter 33 shown in FIGS. 3A-B, as described hereinabove). The guidewire is then passed from inside the subject's chest cavity to outside the subject's chest, through the hole that has been pierced through the chest wall. For some applications, subsequent to the guidewire being passed out of the subject's chest, trocar 40 is inserted between two of the subject's ribs. For example, subsequent to the guidewire being guided out of the subject's chest, the trocar may be inserted through the subject's ribs with a dilator, by the trocar and the dilator being advanced over the guidewire. Alternatively, the remaining steps of the procedure are performed without a trocar being inserted through the subject's ribs. For example, a cannula (typically, cannula 60 described hereinbelow) may be passed over the guidewire from outside the subject's chest into the subject's chest cavity, and through the hole in the apex of the subject's heart, in the absence of a trocar inserted through the subject's ribs.

As a further alternative to performing Steps 4-6, for some applications, grasping element 50 is inserted into the subject's chest cavity in the absence of a trocar. For example, the grasping element may be inserted into the subject's chest cavity via a 4-6 Fr catheter. For some applications, trocar 40 is inserted through the subject's chest wall at a subsequent step of the procedure. For example, subsequent to the guidewire being pulled out of the subject's chest, the trocar may be inserted through the subject's ribs with a dilator, by the trocar and the dilator being advanced over the guidewire. Alternatively, the remaining steps of the procedure are performed without a trocar being inserted through the subject's ribs. For example, a cannula (typically, cannula 60 described hereinbelow) may be passed over the guidewire from outside the subject's chest into the subject's chest cavity, and through the hole in the apex of the subject's heart, in the absence of a trocar inserted through the subject's ribs.

In Step 7 of the procedure, cannula 60 is advanced over guidewire 10, through trocar 40 to the outside of apex 6. In Step 8 of the procedure, the cannula is stabilized with respect to the outer surface of the wall of the heart at the apex by a suction cup 77 being placed against the outer surface of the wall of the heart at the apex and applying vacuum pressure via a suction lumen of the cannula, such as to create a vacuum between the suction cup and the outer surface of the wall of the heart at the apex. Steps 7 and 8 are shown in FIG. 1C. Typically, as shown, in order to facilitate insertion of the suction cup through the trocar, the suction cup is folded during the insertion, and a constraining sheath 78 is placed around the suction cup, such as to maintain the suction cup in the folded configuration. Subsequent to the suction cup having been pushed through the trocar, the suction cup is pushed distally with respect to the constraining sheath, such that the suction cup is no longer constrained by the sheath. The suction cup is configured such that when the suction cup is not constrained by the sheath, the cup automatically unfolds, as described in further detail hereinbelow.

For some applications, subsequent to the suction cup being placed against the outer surface of the wall of the heart at the apex, as described with respect to Step 8, Step 8a of the procedure, namely the opening of a pericardial window, is performed. For example, one or more balloons may be placed between the myocardium and the pericardium, the balloons being coupled to a sharp element and/or an electrode configured for cutting an incision in the pericardium in order to allow passage of fluid therethrough (e.g., a pericardial window), thus preventing accumulation of blood or fluid between myocardium and the pericardium which may lead to pericardial tamponade. For such an application, a scoring balloon may be used, e.g., placed inside or outside cannula 60. The scoring balloon is typically passed through cannula 60 in order to arrive at a location where it is used to create the incision in the pericardium. Alternatively, a balloon coupled to a sharp element and/or an electrode may be placed between the myocardium and the pericardium for creating an incision in the pericardium.

For some applications, instead of or in addition to the electrode or cutting device disposed on a balloon as described hereinabove, a catheter is passed through a hole in a lateral wall of cannula 60, and an electrode or cutting device is passed through the catheter and is used to create an opening in the pericardium, e.g., to prevent tamponade. Alternatively or additionally, the catheter that is used for opening the pericardial window is passed through an incision in the skin that is separate from the incision through which cannula 60 is passed, and the electrode or cutting device is passed through the catheter and creates the opening in the pericardium. Further alternatively or additionally, the catheter that is used for opening the pericardial window is passed through trocar 40, but is a separate device from cannula 60. For example, the catheter that is used to open the pericardial window may be inserted through trocar 40 before cannula 60 is inserted through the trocar. For some applications, the electrode or cutting device that is used for opening the pericardial window is inserted through an introducer sheath, such as a 12-14 Fr introducer sheath.

Typically, subsequent to the suction cup being placed against the outer surface of the wall of the heart at the apex, as described with respect to Step 8, catheter 12 and structure 30 are retrieved from the subject's left ventricle, as shown in FIG. 1C. Alternatively, catheter 12 and structure 30 are retrieved from the subject's left ventricle at a different stage of the procedure, such as between Steps 6 and 7 of the procedure.

Typically, guidewire 10 has a variable stiffness. For example, the guidewire with variable stiffness may be manufactured having (a) a distal portion which is soft, which is initially passed to the heart, and (b) a proximal portion which is stiffer, and which is advanced to the heart after the soft distal portion has already been passed out of the heart, through the hole in the apex. In this context, in the specification and in the claims, “proximal” means closer to the orifice through which the guidewire/tool is originally placed into the body, and “distal” means further from this orifice. In Step 9 of the procedure, guidewire 10 is advanced into cannula 60, such that the stiff, proximal portion of the guidewire is advanced through the hole in the apex and out of the patient's chest through trocar 40. Typically, the stiff, proximal portion of the guidewire is only advanced through the hole in the apex subsequent to suction cup 77 having been placed at the apex and suction having been performed so as to stabilize the distal end of the cannula with respect to the apex, by creating a vacuum between the suction cup and the outer surface of the wall of the heart at the apex. Further typically, before the distal end of the cannula has been stabilized with respect to the apex, only the soft distal portion of the guidewire is passed through the apical hole so as to prevent the myocardium from being damaged by the heart pulsating while the guidewire is disposed in the hole through the apex. Subsequent to the stabilization of the distal end of the cannula with respect to the apex, the distal end of the cannula protects the myocardium from being damaged. Therefore, at this stage, the proximal, stiff portion of the guidewire may be advanced through the hole through the apex. The proximal, stiff portion of the guidewire is typically used to facilitate the guidance of tools (e.g., an inner tube of cannula 60 with dilator 90, described hereinbelow, a Transcatheter Aortic-Valve Implantation (TAVI) introducer sheath with a dilator, and/or an introducer sheath of a prosthetic mitral valve with a dilator) through trocar 40, and/or through the hole in the apex of the subject's heart.

It is further noted that, typically, prior to cannula 60 being stabilized with respect to the apex, generation of tension in the guidewire by pulling on the distal end of the guidewire is avoided. Rather the guidewire is advanced, by the proximal end of the guidewire being fed through the femoral access point, and by the distal end of the guidewire being directed out of the patient's chest by being pulled gently with the grasping element.

Typically, the guidewire having the varied stiffness is formed by using a connection element to connect a first soft guidewire to a stiffer guidewire, and, optionally, an additional connection element is used to connect this stiffer guidewire to an even stiffer guidewire. Typically, but not necessarily, these successive guidewires are connected to each other by means of friction, such as by a crimping tube placed around the two guidewires that are to be coupled together (typically at the time of manufacture). In addition, these connections can typically be easily disconnected, in order to again form two or more distinct guidewires. For some applications, radiopaque markers are disposed on one or both portions of a guidewire having varied stiffness such that the portions of the guidewire are identifiable in fluoroscopic images of the guidewire. For example, radiopaque markers having respective shapes may be disposed on the portions of the guidewire having different levels of stiffness. Or, radiopaque markers having respective spacings between adjacent markers may be disposed on the portions of the guidewire the different levels of stiffness.

For some applications, guidewire 10 defines an outer soft layer, and an inner stiff core that is moveable with respect to the outer soft layer. For example, the guidewire may be generally similar to moveable core guidewires known in the art, such as those manufactured by Cook® Medical. Initially the guidewire is advanced to the apex, while the stiff inner core is retracted with respect to the distal end of the soft outer layer of the guidewire. Thus, at this stage, the distal end of the guidewire is soft and is configured not to cause an injury the inner wall of the heart at the apex. Catheter 12 is guided to the apex by being advanced over the guidewire. When the distal end of catheter 12 is stabilized at the apex, the soft outer layer of the guidewire is retracted into the catheter, such that the inner core of the guidewire stiffens the distal end of the guidewire. Typically, at this stage, the positions of the stiff inner core and the soft outer layer of the guidewire are locked with respect to one another. The distal end of the guidewire is then advanced through the wall of the heart at the apex, such as to penetrate the wall of the heart at the apex. For some applications, the distal end of the guidewire penetrates the wall of the heart at the apex by the distal end of the guidewire itself piercing a hole through the apex. Alternatively, an inner catheter 33 (shown in FIGS. 3A-B) that is disposed inside catheter 12 pierces a hole through the wall of the heart at the apex, and the guidewire penetrates the wall of the heart at the apex by being advanced through the inner catheter, in accordance with the techniques described hereinbelow.

For some applications, guidewire 10 is covered with a soft outer layer, such as a soft, plastic outer layer.

In Step 10 of the procedure, an inner tube 62 of cannula 60 and a dilator 90 (typically disposed within the inner tube) are advanced through the myocardial tissue at apex 6 over guidewire 10 (typically, over the proximal stiff portion of the guidewire, as described hereinabove). The dilator dilates the hole at the apex of the heart by being advanced through the hole over the guidewire. Typically before inserting the cannula into the subject's body the space between the inner tube and the dilator is flushed, by injecting a flushing liquid into the space via a flushing port 69 of the cannula. For some applications, flushing the space between the inner tube and the dilator prevents air emboli being introduced into the subject's bloodstream by the cannula.

Inner tube 62 of cannula 60 is typically advanced through the myocardial tissue by pushing the inner tube distally with respect to an outer tube 64 of the cannula, by pushing a portion 63 of the handle of the cannula distally. A balloon 72 is disposed at the distal end of the inner tube of the cannula. In Step 11 of the procedure, when the distal end of inner tube 62 is disposed inside the subject's left ventricle 4, balloon 72 is inflated by injecting fluid (e.g., a liquid, such as saline) into the balloon via an inflation port 67 of the cannula. Balloon 72 is typically shaped to conform with the shape of the inner wall of the ventricle at the apex, as described in further detail hereinbelow. In Step 12 of the procedure, subsequent to the balloon having been inflated, inner tube 62 is retracted proximally with respect to outer tube 64 of the cannula, such that the balloon is pulled back against the inner wall of the ventricle at the apex. The position of the inner tube 62 with respect to outer tube 64 of the cannula is typically fixed, for example, by locking the positions of the tubes with respect to one another using a locking mechanism 66. Steps 10-12 are shown in FIG. 1D.

As described hereinabove, suction cup 77 is typically placed on the outer surface of the wall of the heart at the apex, and a vacuum is created between the suction cup and the outer surface of the wall of the heart at the apex. Typically suction cup 77 is disposed at the distal end of outer tube 64 of cannula 60. Thus, when inner tube 62 of the cannula is retracted proximally with respect to the outer tube of the cannula, as described above, the tissue of the heart surrounding the hole in the apex is secured (e.g., by being gently squeezed) between balloon 72 and the suction cup. Typically, the vacuum between the suction cup and the outer surface of the wall of the heart at the apex is created by applying vacuum pressure through the space between inner tube 62 and outer tube 64 of cannula 60. Thus, a vacuum is formed between the suction cup and the tissue of the heart surrounding the hole, thereby sealing the suction cup to the tissue that surrounds the hole in the apex. Typically, balloon 72 provides sealing of the cannula 60 with respect to the inner surface of the wall of the heart at the apex.

Subsequent to sealing the tissue of the heart that surrounds the hole with respect to the suction cup and securing the tissue surrounding the hole between the balloon and the suction cup, dilator 90 is withdrawn from the inner tube of cannula 60. At this stage, the inner tube 62 of cannula 60 provides a working channel from outside the subject's chest to inside the subject's heart, via the hole in the apex of the heart. The distal end of the cannula is sealed with respect to the tissue that surrounds the hole at the apex, as described hereinabove. For some applications, a further tube that is disposed within inner tube 62 of the cannula provides the working channel of the cannula. FIG. 1E shows the subject at this stage in the procedure. As shown, guidewire 10 passes into the subject's body through a peripheral artery (e.g., the femoral artery, as shown) and back out of the subject's body through the patient's chest. Cannula 60 provides a working channel from outside the subject's chest, through trocar 40, and into the subject's heart via the hole in the apex.

In Step 13, a cardiac interventional procedure is performed with respect to the subject's heart, using the working channel that has been created through the patient's chest into the subject's heart via the hole at the apex. Typically a working catheter that is used to perform the procedure is inserted into the subject's heart via the working channel. For example, a valve of the subject's heart may be repaired or replaced (e.g., the subject's aortic valve may be replaced using a TAVI procedure, and/or the subject's mitral valve may be replaced), or a different cardiac structure may be repaired or replaced. Alternatively or additionally, the working channel can provide access to the subject's heart to facilitate any other type of cardiac surgery that can be performed with a minimally-invasive approach, such as ablation of a heart wall, implantation of a cardiac assist device, repairing a structural defect of the heart, repair of a failed bioprosthesis, treatment of atrial fibrillation, and/or transvascular approach to repairing or implanting a device in the ascending aorta, the aortic arch, and or the carotid arteries. For some applications, two or more guidewires pass through the working channel. For example, guidewire 10 may be used to facilitate an aortic intervention, and an additional guidewire may be introduced from the apex into the left atrium, via the working channel, in order to facilitate a mitral intervention.

In Step 14, subsequent to performing the cardiac interventional procedure, the tools that were used to perform the procedure are withdrawn from the working channel of cannula 60.

In Step 15, a hole closure device 80 is advanced through the working channel of cannula 60. The hole closure device typically defines an intracardiac portion 81, a plug portion 82, and an extracardiac portion 83, as described in further detail hereinbelow with reference to FIG. 1F. The hole closure device is pushed such that the intracardiac portion thereof protrudes from the distal end of cannula 60 inside the subject's heart (typically, inside the subject's left ventricle). The hole closure device is typically configured such that, upon protruding from the distal end of the cannula, the intracardiac portion assumes a concave shape that conforms with the shape of the inner wall of the heart at the apex.

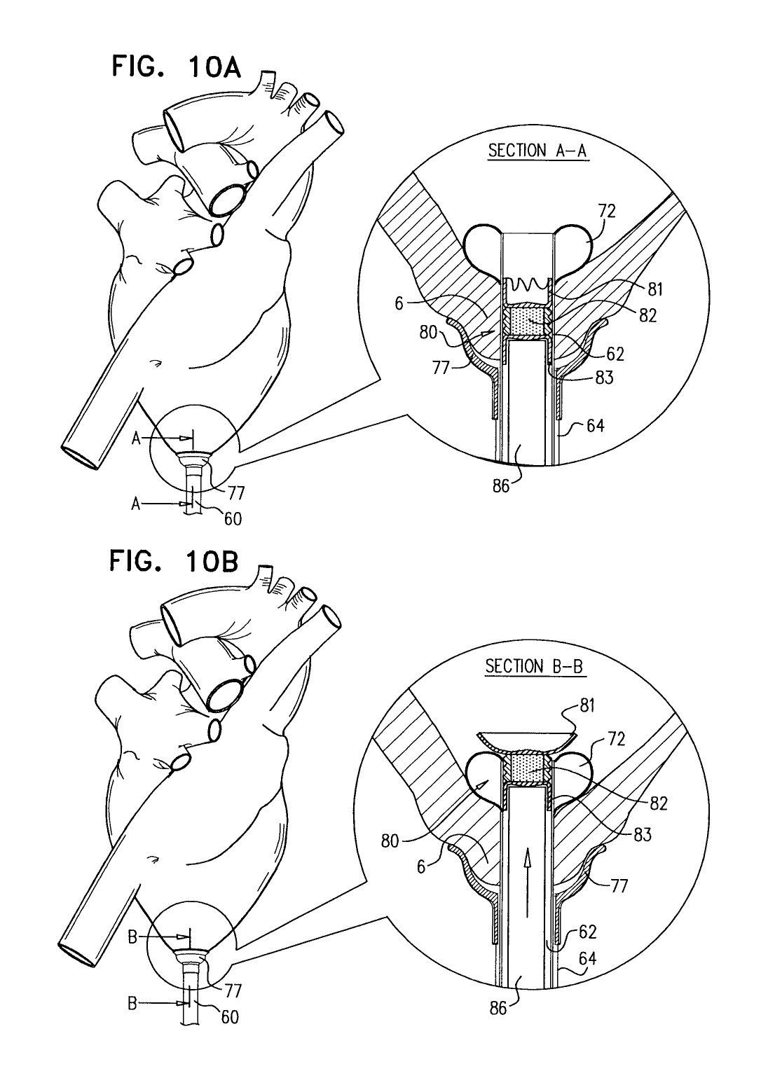

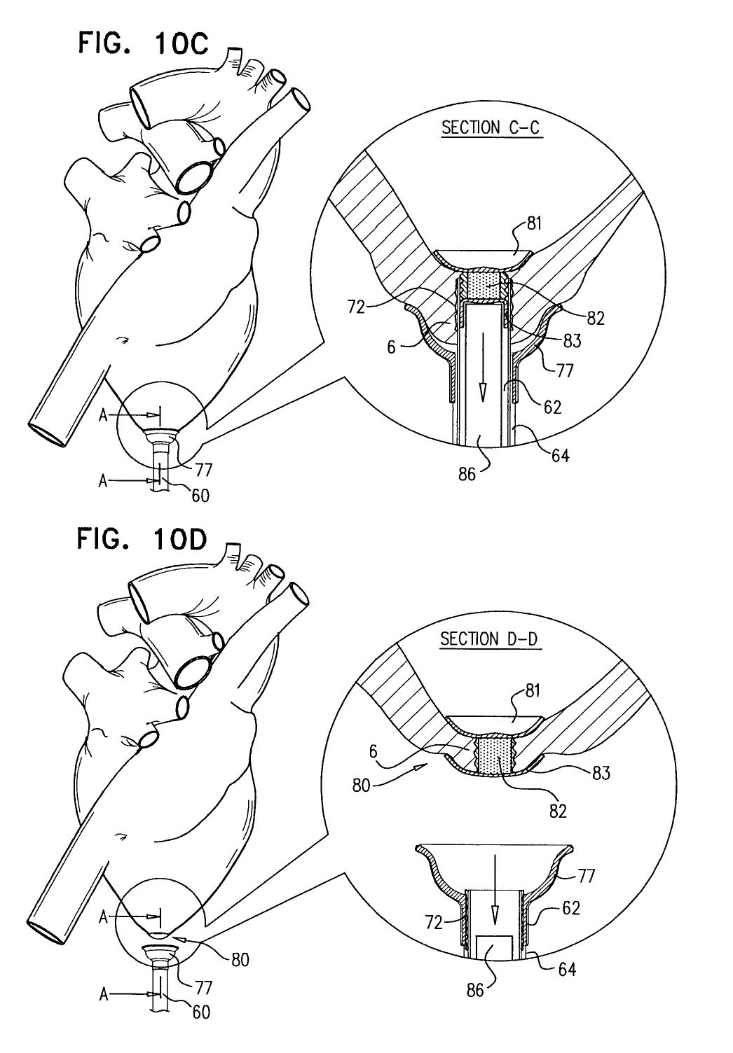

In Step 16, balloon 72 is deflated and inner tube 62 of cannula 60 is retracted. The retraction of the inner tube of the catheter pulls the intracardiac portion of hole closure device 80 against inner wall of the apex of the subject's heart. Subsequently, the inner tube of the cannula is further retracted, such as to release plug portion 82 of the hole closure device from the inner tube of the cannula. For example, a pushing element 86 (shown in FIGS. 10A-D) disposed within inner tube 62 may be configured to hold the hole closure device stationary with respect to the subject's heart while the inner tube of the cannula is retracted. Pushing element 86 typically defines a lumen. The pushing element is advanced through inner tube 62 over the guidewire, the guidewire passing through the lumen. The plug portion of the hole closure device is configured to automatically expand, such as to fill, and thereby form a plug, within the hole in the apex, as described in further detail hereinbelow.

In Step 17, suction of suction cup 77 is terminated and outer tube 64 and inner tube 62 of cannula 60 are retracted from the subject's heart and out of the subject's chest through trocar 40. The retraction of the cannula is such as to cause extracardiac portion of hole closure device 80 to be released from inner tube 62. For example, a pushing element disposed within inner tube 62 may be configured to hold the hole closure device stationary with respect to the subject's heart while the cannula is retracted. The hole closure device is typically configured such that, upon protruding from the distal end of the cannula, the extracardiac portion assumes a concave shape that conforms with the shape of the outer surface of the wall of the apex of the heart. In general, the hole closure device is configured to automatically seal the hole in the apex, subsequent to the removal of cannula 60 from the hole, as described in further detail hereinbelow.

In Step 18, guidewire 10 and trocar 40 are removed from the subject's body. The guidewire is removed by pulling the guidewire from the proximal end of the guidewire (e.g., at the subject's femoral artery), or from the distal end of the guidewire (at the subject's chest). FIG. 1F shows the subject's heart at this stage. As shown, cannula 60 has been removed, and hole closure device 80 is disposed in the hole in the apex of the heart, such as to seal the hole in the apex. As shown the intracardiac and extracardiac portions of the hole closure device have concave shapes that conform respectively with the inner and outer surfaces of the wall of the heart at the apex.

Reference is now made to FIGS. 3A-C, which are schematic illustrations of femorally-inserted or radially-inserted catheter 12 located in left ventricle 4, and of protective, fixation, and/or locating structure 30, which is coupled to a distal portion of the catheter, located against the apex of the left ventricle, in accordance with some applications of the present invention. In this context, in the specification and in the claims, “proximal” means closer to the orifice through which a tool is originally placed into the body, and “distal” means further from this orifice. Typically, structure 30 facilitates placement of the distal end of the catheter at the apex, since the structure is shaped to conform with the intracardiac side of the apex. Further typically, structure 30 stabilizes the distal end of the catheter at the apex. Still further typically structure 30 provides sealing of the hole that is pierced through the apex. For some applications, structure 30 is configured to reduce the possibility that the distal portion of the catheter may cause undesired damage to the interior of the heart chamber. For some applications, structure 30 is coupled to a shaft of catheter 12 in a compressed state thereof while the catheter is being advanced towards the heart of the subject. Structure 30 is typically configured to expand upon entry to a desired body lumen, e.g., the heart chamber.

Typically, structure 30 comprises an inflatable and/or an expandable element, such as a balloon (shown in FIGS. 3A-C) that inflates upon entry to the heart chamber. When inflated, the balloon typically protects the ventricle, and/or helps to seal a puncture site during the procedure. Typically, the balloon is coupled to a distal end of catheter 12. Structure 30 is expanded within left ventricle 4 and positioned against the apex wall from within the left ventricle. Structure 30 is typically configured such that in an inflated state thereof the distal end of the balloon is distal to a tip 122 of catheter 12. Thus, positioning of the balloon against the apex allows maneuvering space of distal tip 122 within a protected defined space created by the balloon. For some applications, the balloon is shaped to define bulges 31 on the outer surface of the balloon. The bulges are configured to generate friction between the outer surface of the balloon and the inner wall of the heart at the apex, thereby stabilizing the balloon and the distal tip of the catheter at the apex.

For some applications, distal tip 122 of catheter 12 is steerable. Alternatively or additionally, the distal tip defines a channel therethrough, the longitudinal axis of at least a distal portion of the channel being disposed at an angle from the local longitudinal axis of the catheter. For some applications, the distal tip is configured to direct guidewire 10 out of the distal tip of the catheter at an angle from the local longitudinal axis of the catheter, by the guidewire being directed out of the channel defined by the distal tip.

For some applications, first catheter 12 is advanced over a first, typically flexible guidewire, e.g., a 0.089 cm (0.035 inch) soft wire known in the art, into a chamber of the heart, e.g., left ventricle 4. Catheter 12, e.g., a 3-9 Fr catheter, is advanced over the guidewire to a desired location within the chamber of the heart, e.g., against apex 6 of the left ventricle 4. For some applications, structure 30 may provide guidance for catheter 12 to position the catheter at the apex, and/or may be used in order to reduce possible damage to the apex when catheter 12 is positioned against the apex. For some applications, an inner catheter 33 (shown in FIGS. 3A-B), typically smaller in diameter than the first catheter, e.g., a 3-5 Fr catheter, is passed over the first guidewire and through the first catheter. Typically, the inner, small diameter, catheter 33 comprises a sharp distal end configured to puncture and penetrate the heart wall. The inner catheter punctures the heart wall at the apex such as to create a passage through the apex, as shown in FIG. 3A. Guidewire 10 is passed through the passage in the apex, via the inner catheter, as shown in FIG. 3B. Subsequently, the distal portion of the guidewire is advanced out of the subject's chest, as described hereinabove. Typically, subsequent to the inner catheter having created a passage through the apex and the guidewire having been passed through the passage, the inner catheter is retracted into first catheter 12, such that the guidewire remains in the subject's chest cavity in the absence of the inner catheter, as shown in FIG. 3C. For some applications, a distal portion of the inner catheter is advanced out of the subject's chest together with the distal portion of the guidewire. Optionally, the inner catheter is steerable and is used to steer distal portions of the inner catheter and the guidewire toward the chest wall. It is noted that for some applications, inner catheter 33 is a hollow needle, for example a nitinol or a stainless steel hollow needle.

For some applications, after establishing the passage through the apex to the skin, the first flexible guidewire is transfemorally removed, and a second guidewire (e.g., guidewire 15, shown in FIG. 3H) is transfemorally introduced, typically less flexible than the first, e.g., a stiff or super stiff 0.089 cm (0.035 inch) wire, for passage of tools into the heart. For some applications, a single guidewire having a stiffness that varies along the length of the guidewire (e.g., having a distal flexible portion, and a proximal, stiff portion) is used, as described hereinabove, rather than using first and second guidewires.

For some applications, distal tip 122 of catheter 12 is flexible. For example, the tip may include a compliant material which is configured to reduce the possibility that the distal portion of the catheter may cause damage to the interior of the heart chamber. For some applications, such a configuration of distal tip 122 does not require the use of an additional protective structure 30. Thus, for some applications, catheter 12 is inserted into the subject's heart (e.g., toward the inner surface of the heart at the apex) in the absence of structure 30, the catheter defining a flexible distal tip thereof. Alternatively, structure 30 is used to protect the interior of the heart chamber (and/or to provide one or more of the additional functionalities of structure 30, such as to facilitate placement and stabilization of the distal tip of the catheter at the apex) in addition to the tip of the catheter being flexible. It is noted that in general, any of the components placed in the patient's body may include one or more radiopaque portions (e.g., radiopaque markers), e.g., a tip portion of catheter 12 may be radiopaque.

For some applications, distal tip 122 of catheter 12 is composed of silicone or any other suitable flexible and compliant material, e.g., latex and/or polyurethane. Typically distal tip 122 is 0.3-10 mm in length, e.g., 0.5-6 mm. For some applications, a portion of catheter 12 between the distal tip and the proximal portion of catheter includes a stiff elastomer or other suitable plastic material, which is typically intermediate in a mechanical property (e.g., stiffness) and/or structural behavior between that of the distal tip and that of the proximal portion of the catheter.

Reference is now made to FIGS. 3D-E, which are schematic illustrations of protective, fixation, and/or locating structure 30, in accordance with some applications of the present invention. For some applications, structure 30 includes a mesh (shown in FIG. 3D) that can be enlarged or enlarges automatically from a constrained configuration to an unconstrained configuration. Alternatively or additionally, structure 30 includes a shape memory element (shown in FIG. 3E) that can be enlarged or enlarges automatically from a constrained configuration to an unconstrained configuration. As shown in FIG. 3E, for some applications, structure 30 comprises a three-dimensional structure comprising two or more nitinol or stainless steel wires (or another material) which can be deployed and/or expanded in the heart chamber. For some applications, the protective structure is disposed symmetrically with respect to the longitudinal axis of catheter 12, as shown. Alternatively, the protective structure is disposed asymmetrically with respect to the longitudinal axis of catheter 12 (application not shown). For some applications, combinations of two or more of the various protective structure described herein are used. It is noted that, in general, examples described herein relating to use of an inflated balloon may be, in each case, carried out using another type of expandable element, such as (a) a mesh (shown in FIG. 3D) that can be enlarged or enlarges automatically from a constrained configuration to an unconstrained configuration, or (b) a shape memory element (shown in FIG. 3E) that enlarges from a constrained configuration to an unconstrained configuration

For some applications, protective structure 30 includes any suitable three-dimensional structure, e.g., a balloon or a braided mesh, comprising nitinol or stainless steel or cobalt chromium (or another material) which can be deployed and/or expanded in the heart chamber. Typically, the protective structure provides support for the stabilization of the catheter. Additionally or alternatively, protective structure 30 advanced transvascularly into the left ventricle serves as a radiopaque marker for locating the left ventricle and any element used during the procedures for transthoracic cardiac surgery described herein. For some applications, the protective structure is shaped to fit snugly in the apex, in order to provide stabilization of catheter 12 as described, and/or to facilitate proper subsequent creation of a hole in the apex.

Typically, protective structure 30 reduces the possibility that the distal portion of the catheter may cause damage to the interior of the heart chamber. Additionally or alternatively, protective structure 30 provides support for the stabilization of catheter 12. Further additionally or alternatively, protective structure 30 provides guidance for the catheter, by facilitating proper positioning of the distal portion of catheter 12 against apex 6. In particular, use of protective structure 30 facilitates guidance of a piercing element (e.g., inner catheter 33, or second guidewire 15, described hereinabove) to a desired puncture site in the apex.

Reference is made to FIGS. 3F-H, which are schematic illustrations of the advancement of a second guidewire 15 (shown in FIG. 3H) out of the apex of the left ventricle, in accordance with some applications of the present invention. For some applications, first guidewire 10 (shown in FIG. 3F) is a flexible guidewire such as a flexible 0.089 cm (0.035 inch) wire or a 0.08 cm (0.032 inch) wire, which, in the absence of any external forces, has a soft curved distal portion 11.

For some applications, catheter 12 is a 4-9 Fr multi-lumen or single-lumen catheter. As described hereinabove, catheter 12 is advanced over guidewire 10 to a desired location within the left ventricle, e.g., against apex 6 of the left ventricle 4. In accordance with respective applications, catheter 12 may or may not have variable stiffness along the length thereof. For some applications, catheter 12 has a curved distal portion 13 comprising a distal tip shaped to define a flexible “J” (or alternatively the tip is pigtail-shaped). The distal portion of catheter 12 typically fits into the naturally curved anatomical shaped of the apex of the left ventricle as shown in FIG. 3G. Typically, the distal portion of catheter 12 comprises an aperture portion. The aperture portion is shaped to define one or more apertures 14. Aperture 14 typically covers ⅛ to ¾ of the circumference of catheter 12. For some applications, at least one of the apertures is used to provide suction, e.g., in order to stabilize catheter 12 against the inner surface of the left ventricle.

Subsequently to positioning of catheter 12 against the apex of the left ventricle, first guidewire 10 is removed and a second, typically less flexible, guidewire 15 is advanced through catheter 12 to the apex of the left ventricle. The second guidewire typically comprises a sharp distal portion (not shown) configured to puncture the apex of the heart from inside the left ventricle. The second guidewire is advanced to aperture 14 in catheter 12 and is advanced through the aperture to puncture and penetrate the wall of the left ventricle at the apex, as shown in FIG. 3H. Typically, the second guidewire is then advanced towards skin of the subject as described herein. (For some applications, catheter 12 is shaped to define multiple apertures 14, and the second guidewire is advanced to a suitable one of the apertures.)

Typically, the distal tip of catheter 12, which is shaped to define a “J”, has a radius of curvature that is between 2 and 40 mm, e.g., between 5 and 20 mm. The distance between aperture 14 and the distal tip is typically between 1 and 40 mm, e.g., between 10 and 25 mm (as measured along the length of catheter 12). Such a configuration of catheter 12 typically conforms to apical anatomy and enables performing procedures described herein in a repeatable and reproducible manner.

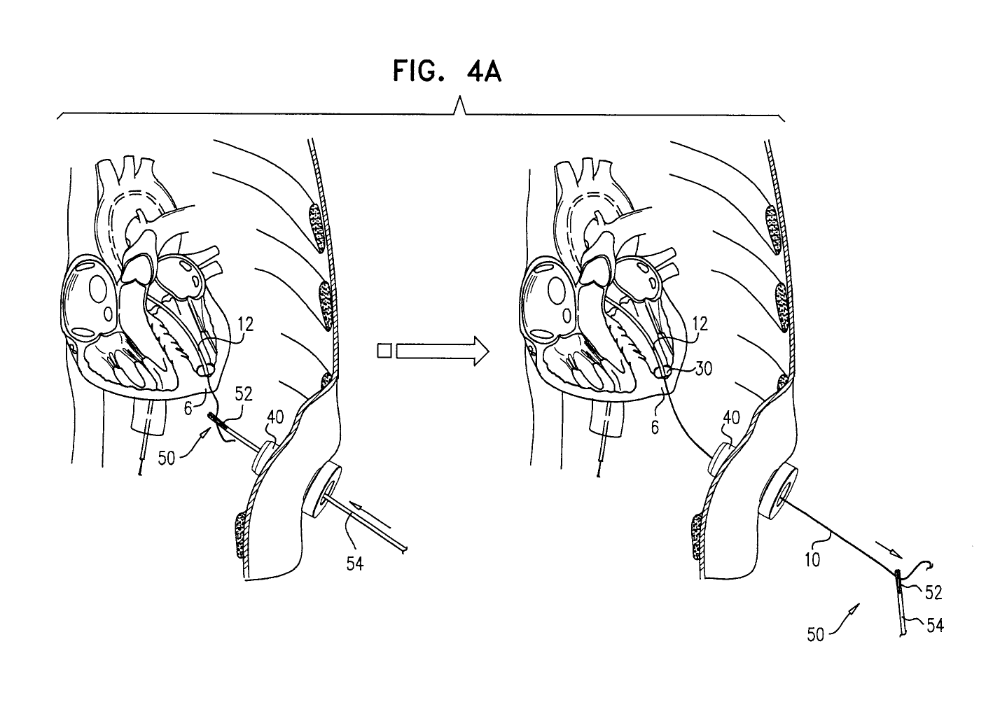

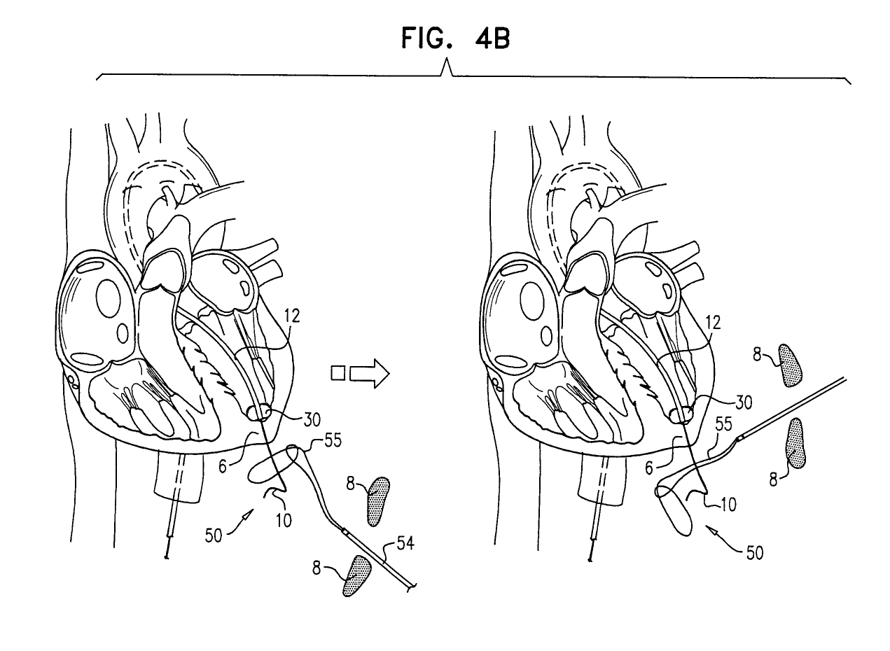

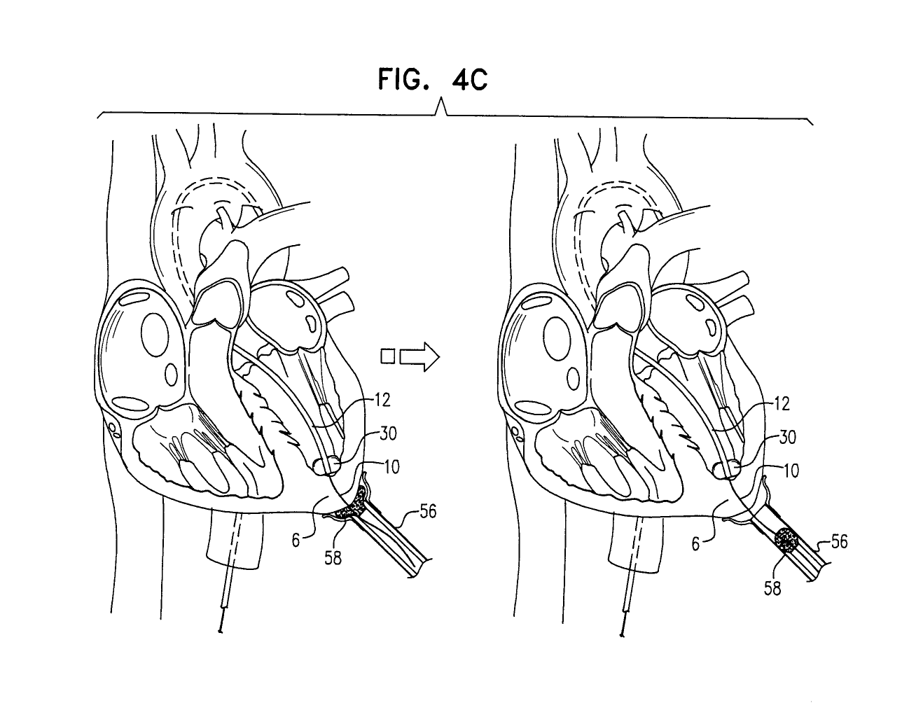

Reference is now made to FIGS. 4A-C, which are schematic illustrations of grasping devices 50 for receiving and directing guidewire 10 upon exiting apex 6, in accordance with some applications of the present invention. For some applications, grasping element 50 includes a hinged clip 52 disposed at the end of an elongate insertion rod 54, as shown in FIG. 4A. The insertion rod is inserted into the subject's chest cavity, via trocar 40, and the clip is used to grasp the distal end of guidewire 10. The elongate element is then pulled out of the subject's chest cavity via the trocar, such as to direct the distal end of the guidewire out of the subject's chest. Structure 30 in left ventricle 4 typically serves as a guide for locating the left ventricle and for proper positioning of clip 52 on the external side of the apex. As described hereinabove, typically, prior to cannula 60 being stabilized with respect to the apex, generation of tension in the guidewire by pulling on the distal end of the guidewire is avoided. Rather the guidewire is advanced, by the proximal end of the guidewire being fed through the femoral access point, and by the distal end of the guidewire being directed out of the patient's chest by being pulled gently with the grasping element.

As shown in FIG. 4B, for some applications, grasping element 50 includes a snare 55, which is advanced towards the heart of a subject through ribs 8. Snare 55 is typically disposed at the end of elongate insertion rod 54, which is generally as described with reference to FIG. 4A. Snare 55 typically comprises a cable or wire which is configured to engage guidewire 10 and direct the guidewire in a desired direction, e.g., towards skin of the subject. Guidewire 10 may be engaged by advancing snare 55 in a longitudinal direction, as shown on the left of FIG. 4B, or in a lateral direction, as shown on the right of FIG. 4B. Guidewire 10 then establishes a path between the heart, for example, the left ventricle, and skin of the subject.

As shown in FIG. 4C, for some applications, following deployment of catheter 12 and protective structure 30 in left ventricle 4, an incision is made in the chest wall and a catheter 56 is advanced towards the external side of the apex 6. Protective structure 30 in left ventricle 4 typically serves as a guide for locating the left ventricle and for proper positioning of catheter 56 on the external side of the apex. Catheter 56 may comprise a 4-14 Fr (e.g., 6-12 Fr) catheter with a wide, optionally expandable, soft tip to interface with the external side of the apex. For some applications, cannula 60 is used as catheter 56, suction cup 77 of cannula 60 functioning as a soft tip for interfacing with the external side of the apex.

For some applications, grasping element 50 includes an apical element 58, which is advanced through catheter 56 and positioned against the external side of the apex. Element 58 may comprise a braided mesh or any other suitable configuration of nitinol and/or stainless steel and/or plastic or other material, suitable for grasping or otherwise holding a tool such as a guidewire. Element 58 is typically positioned against the external side of the apex and may apply slight pressure to the apex for stabilization of catheter 12, located in the left ventricle.

For some applications, guidewire 10 is then removed from catheter 12, and a second guidewire (e.g., guidewire 15, shown in FIG. 3H) is advanced through catheter 12 into left ventricle 4. For some applications, as described hereinabove, only guidewire 10 is used, and guidewire 10 has a first, distal, flexible portion and a second, proximal, stiff portion. The second guide wire (or second portion of guidewire 10) is typically less flexible than the first guidewire (or first portion of guidewire 10) and may comprise a stiff or super stiff 0.089 cm (0.035 inch) wire. For some applications, the second guidewire comprises a pointed steerable (or non-steerable) tip, configured to puncture a wall of the heart from within the chamber of the heart and create a passage in the wall of the heart. The second guidewire, or the second portion of guidewire 10 is typically passed out of catheter 12 and through the passage in the apex (the passage may be created by the second guidewire, or alternatively by any other appropriate puncturing device, e.g., inner catheter 33 shown in FIGS. 3A-B). Typically, protective structure 30 provides support for the guidewire while it is being advanced out of the left ventricle and through the passage in the apex. Additionally, structure 30 is deployed within the heart chamber such that it guides the guidewire towards a desired exiting location from the apex.

Guidewire 10, or second guidewire 15 is passed out of the heart and into catheter 56, where it is received by element 58, which is positioned within catheter 56 and against the external side of the apex. Element 58 typically facilitates guiding of the guidewire into the catheter 56. For some applications, element 58 includes a magnet which is configured to take hold of the guidewire and direct it into catheter 56. For some applications, hinged clip 52, described with reference to FIG. 4A is inserted via catheter 56 and is used to guide the guidewire through catheter 56 in a desired direction. For some applications, element 58 is retracted after guiding the guidewire into catheter 56.

Guidewire 10 or second guidewire 15 continues to be advanced through catheter 56 to the skin of the subject, such that the guidewire or a portion thereof extends from the heart to the skin, establishing a path. For some applications, the skin includes skin of a chest of the subject, and the guidewire is passed through the chest wall to the skin of the chest. For some applications, the guidewire is advanced through the passage and a short distance towards skin of the subject. At that point, the guidewire may be guided, e.g., by use of an additional tool, out of the passage and towards skin of the subject. Alternatively, the guidewire is advanced through the passage in the heart wall and through a portion of the distance towards the skin and subsequently directed toward the skin by use of an additional tool. Guidewire 10 or 15 then establishes a path between the heart, specifically the left ventricle, and skin of the subject.

In some applications, first guidewire or second guidewire 15 comprises proximal and distal ends thereof, and the distal end typically has a straight configuration while being passed through catheter 12. For some applications, the distal tip of the guidewire comprises needle functionality and is configured to puncture a wall of the heart chamber from within the chamber of the heart and create a passage in the wall of the heart. The guidewire is then passed through the passage, out of the heart. For some applications (not shown), the distal end of the guidewire assumes a curved configuration upon exiting the heart chamber, such that the distal end is curved towards skin of the subject, and generally away from the patient's diaphragm. For some applications, the curved guidewire is rotated to a desired position, e.g., towards skin of the subject. The guidewire may be rotated under fluoroscopy or any other suitable imaging means, or alternatively without any imaging. The guidewire is then advanced towards and through the skin of the subject, such that the guidewire extends from the heart to the skin, establishing a path. Typically, a curved distal end of the guidewire allows for more precise directing and advancing of the guidewire towards skin of the subject, and generally reduces the risk of the guidewire penetrating abdominal organs in its vicinity.

For some applications, catheter 12 itself is configured to mechanically maintain curved first guidewire 10 or second guidewire 15 in a straight configuration while inside the catheter. For some applications, the guidewire comprises a shape memory material, e.g., nitinol and/or stainless steel, or elgiloy, or any cobalt-chromium wire, such as MP35N, or any other suitable material known in the art. For some such applications, the guidewire may comprise nitinol and may be cooled inside the catheter and thereby be deformed into a straightened (substantially not curved) configuration. Upon exiting the catheter, the nitinol guidewire reaches body temperature, causing it to regain its original curved shape.

For some applications, a magnet is applied to the skin surface from outside the body of the patient and is used to facilitate rotation and steering of first guidewire 10 or second guidewire 15 towards the skin surface of the subject. In this case, the guidewire comprises a magnetic material.

For some applications, first guidewire 10 or second guidewire 15 may preliminarily be passed out of the distal end of the catheter inside the heart chamber. The guidewire, particularly the curved distal end, is then examined, e.g., by fluoroscopy, to determine if it is oriented in a desired direction. The guidewire may then be rotated inside the heart chamber until it reaches a desired orientation. Alternatively, a marking on the guidewire at the femoral artery (or other artery) entrance point is used to indicate the rotational disposition of the curved distal end of the guidewire, within the heart chamber. For example, the marking may be placed on the guidewire, such that when the guidewire is rotated to place the marking in an anterior position with respect to the patient's body, the curved distal end of the guidewire is correspondingly aimed anteriorly, i.e., towards the patient's chest wall.

In summary, in applications described hereinabove, guidewire 10 or second guidewire 15 with or without a curved distal tip is advanced from the heart to the skin of the subject in order to establish a path between the heart (for example, the left ventricle or another chamber), and skin of the subject.

For some applications, subsequently to extending guidewire 10 or guidewire 15 between the heart and the skin of the subject, a dilator is passed from the skin over the guidewire, in order to create an enlarged path to the heart. The enlarged path to the heart facilitates passage of tools used in cardiac procedures, such as valve repair and/or replacement tools (for example, for implantation of a prosthetic aortic valve and/or a prosthetic mitral valve).

For some applications, a plurality of successively larger dilators are passed from the skin over guidewire 10 or guidewire 15, and are used to dilate the path to a suitable size to facilitate the passage of tools through the now-enlarged path, into the heart chamber. For example, a series of concentric dilators may be passed over the guidewire. (As appropriate, smaller dilators may be removed after larger ones have been passed over the guidewire, or they may remain in place.) Alternatively or additionally, an expandable dilator may be used to enlarge the path, e.g., by balloon inflation of the dilator. Optionally, the dilator may be plastically deformable during the inflation, so as to maintain the enlarged path and thereby facilitate subsequent passage of a tool therethrough. For some applications, other techniques (e.g., as are known in percutaneous nephrostomy) are used to dilate the path.

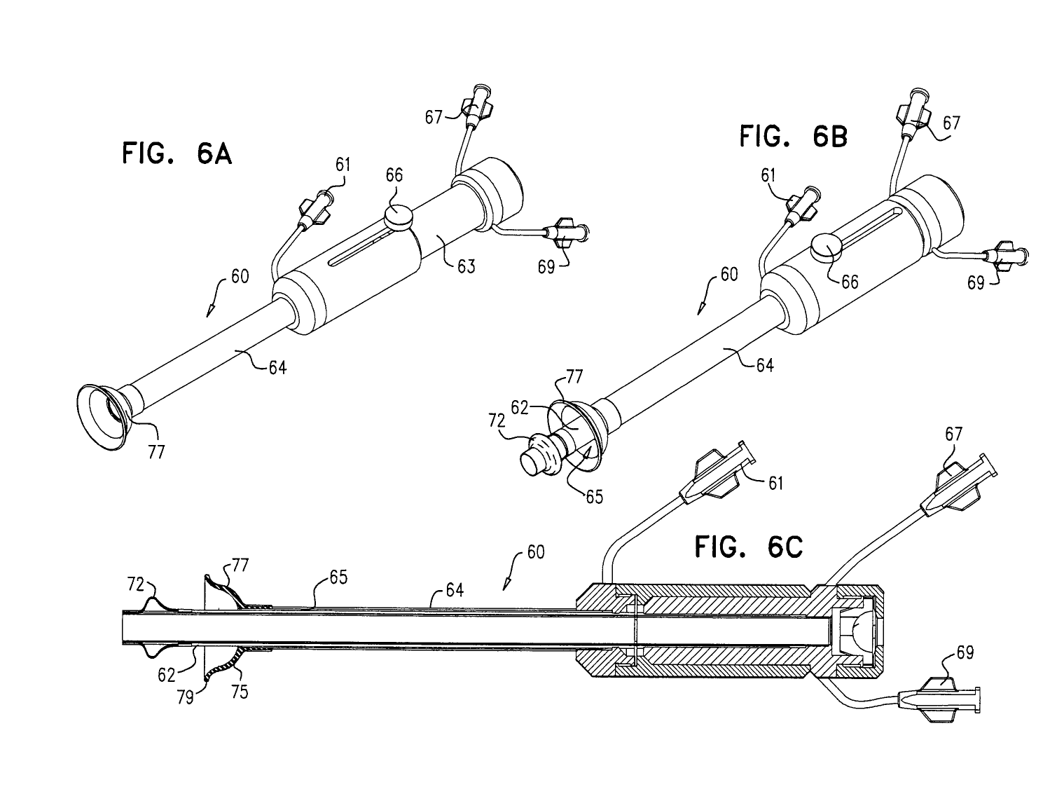

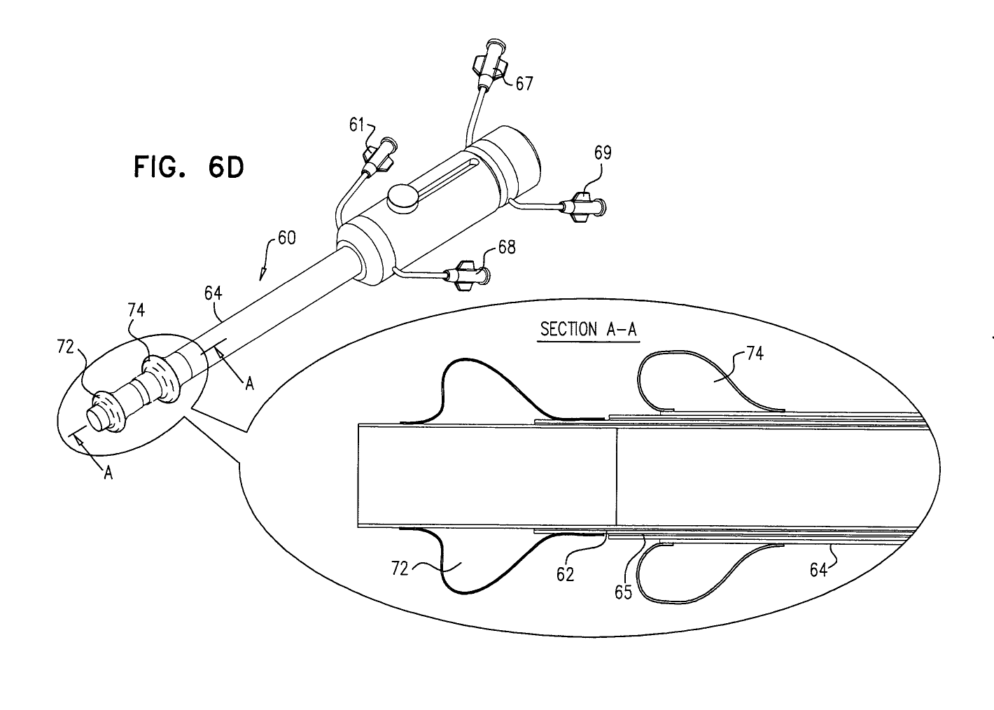

Typically, the enlarged path between the subject's skin and the subject's heart (via the hole in the apex of the heart) is provided by cannula 60 described hereinabove with reference to FIGS. 1-2, and hereinbelow with reference to FIGS. 6A-D.

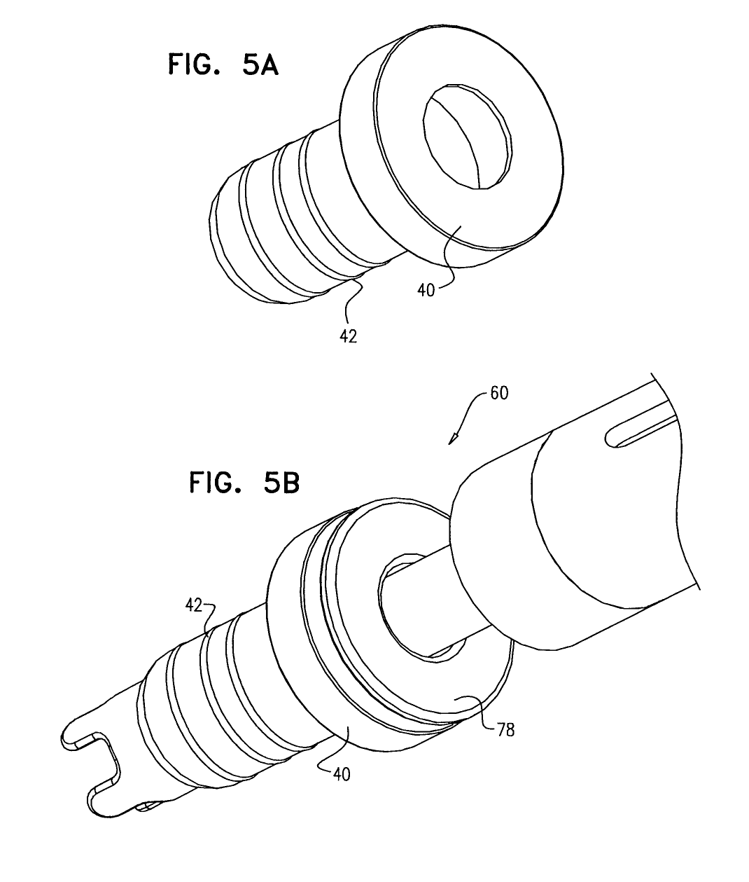

Reference is now made to FIGS. 5A-B, which is a schematic illustration of a trocar 40 configured to be placed between a subject's ribs, in accordance with some applications of the present invention. For some applications, a portion of trocar 40 includes threading 42 on an outer surface thereof, as shown in FIGS. 5A-B. The trocar is advanced between the subject's ribs by the trocar being screwed through an opening in the subject's skin. For some applications, advancing the trocar in this manner facilitates gradual advancement of the trocar. For some applications, threading 42 facilitates anchoring of trocar 40 to soft tissue in the vicinity of the opening in the subject's skin through which the catheter is inserted. For some applications, a proximal portion of another of the tools described herein (e.g., cannula 60) is threaded in order to facilitate the gradual advancement of the tool toward the subject's heart.

FIG. 5B shows trocar 40 while the distal portion of cannula 60 is being advanced through the trocar. As shown, for some applications, during the insertion of the distal portion of the cannula through the trocar, protective sheath 78 is disposed around the distal portion of the cannula. Typically, suction cup 77 is disposed on the distal portion of the cannula, as described herein. Further typically, the suction cup is maintained in a folded configuration by the protective sheath, during the insertion of the distal portion of the cannula through the trocar. The protective sheath thus facilitates passage of the suction cup through the trocar. Subsequent to being passed through the trocar, the distal portion of the cannula is pushed distally with respect to the distal tip of the protective sheath, such that the protective sheath no longer maintains the suction cup in the folded configuration. The suction cup is typically configured to assume a curved shape that is configured to conform with the shape of the outer surface of the heart at the apex, when the suction cup is in a non-constrained state thereof. For some applications, a single structure functions both as (a) trocar 40 to facilitate insertion of cannula 60 through the subject's ribs, and (b) as protective sheath 78 to facilitate the insertion of the suction cup 77 in a folded configuration.

Reference is now made to FIGS. 6A-C, which are schematic illustrations of cannula 60 that is typically inserted through trocar 40 and through a hole in the apex of the subject's heart, in accordance with some applications of the present invention. It is noted that, for some applications, the cannula is inserted between the subject's ribs and through the hole in the subject's apex, in the absence of trocar 40.

As described hereinabove, cannula 60 typically includes inner tube 62 and outer tube 64, the inner tube being slidable with respect to the outer tube. Outer tube 64 forms a passage from the subject's skin to the subject's heart, and inner tube 62 passes into a chamber of the heart through the outer tube, thereby providing a passage from the subject's skin to the subject's heart. Balloon 72 is typically disposed on the distal end of the inner tube, the balloon being configured to be disposed within the heart chamber and to be pulled back against the inner surface of the wall of the heart. Suction cup 77 is typically disposed on the distal end of outer tube 64. Typically, the suction cup defines a flared distal end of outer tube 64. The cannula typically includes one or more (e.g., two or three) hemostatic valves at the proximal end of the cannula. The cannula typically defines vacuum port 61, inflation port 67, and flushing port 69, having functionalities as described hereinabove.

Inner tube 62 and outer tube 64 are typically movable with respect to one another, as described hereinabove. It is noted that, typically, cannula 60 is moveable with respect to trocar 40. Thus, inner tube 62 of cannula 60, and outer tube 64 of cannula 60, are movable with respect to one another and with respect to trocar 40. For some applications, outer tube 64 is advanced toward the heart while inner tube 62 is retracted such that the inner tube is disposed inside the outer tube. Typically during the insertion of the distal end of the outer tube of the cannula through the trocar, the suction cup is folded inside sheath 78 (shown in FIG. 5B). As described hereinabove, subsequent to being passed through the trocar, the distal portion of the outer tube of the cannula is pushed distally with respect to the distal tip of the protective sheath, such that the protective sheath no longer maintains the suction cup in the folded configuration. The suction cup is typically configured to assume a curved shape that is configured to conform with the shape of the outer surface of the heart at the apex, when the suction cup is in a non-constrained state thereof. For some applications, the suction cup includes a shape memory material that is configured to cause the suction cup to assume the curved shape, when the suction cup is in a non-constrained state thereof. For example, the suction cup may include ribs that are made from a shape memory alloy, such as nitinol. For some applications, the ribs are configured to prevent the suction cup from folding backward upon being pushed against the outer surface of the subject's heart. For some applications, the suction cup is configured to assume a curved shape in which a proximal portion 75 of the suction cup is concave in the distal direction (i.e., the direction that is toward the heart and away from the skin of the chest), and a distal portion 79 of the suction cup is convex in the distal direction, as shown in FIG. 6C. For some applications, using a suction cup having a curved shape as described prevents the suction cup from folding over itself when the suction cup is pushed against the outer surface of the wall of the heart. For some applications, the distal edge of the suction cup (i.e., the edge of distal portion 79) is thickened with respect to the rest of the suction cup.

FIG. 6A shows cannula 60 in the absence of the subject's anatomy, as the cannula is configured when the suction cup has been placed against the outer surface of the wall of the subject's heart, and before inner tube 62 has been advanced out of the distal end of outer tube 64. Subsequent to the placement of the suction cup against the outer surface of the wall of the heart, a vacuum is typically formed between the suction cup and the outer surface of the wall of the heart by suctioning fluid from between the suction cup and the outer surface of the wall of the heart. The suction is typically applied via vacuum port 61 that suctions fluid via a space 65 (shown in FIGS. 6B and 6C) between inner tube 62 and outer tube 64.

Subsequent to the sealing of suction cup 77 against the outer surface of the wall of the heart at the apex, the distal end of inner tube 62 is typically slid distally with respect to outer tube 64, for example, by pushing portion 63 of the cannula handle distally. The distal end of the inner tube is advanced to inside the subject's heart via the hole that has been pierced through the apex (i.e., the passage through the apex). Typically, while the distal end of the inner tube is advanced through the passage in the apex, dilator 90, which is disposed inside the inner tube, dilates the passage through the apex, as described hereinabove with reference to FIG. 1D. When the distal end of the inner tube is disposed inside the subject's heart, balloon 72 is inflated. The inflated balloon is then pulled toward the suction cup by moving inner tube 62 proximally with respect to outer tube 64, thereby securing the wall of the heart between balloon 72 and suction cup 77. For some application, balloon 72 is first inflated and pulled against the inner surface of the wall of the heart. Subsequently, suction cup 77 is pushed toward the balloon by pushing outer tube 64 distally with respect to inner tube 62, thereby sandwiching the wall of the heart between balloon 72 and sealing portion 77. For some applications, when the wall of the heart is secured between balloon 72 and suction cup 77, the position of the inner tube with respect to the outer tube is locked using locking mechanism 66. Typically, the vacuum between the suction cup and the outer surface of the wall of the heart at the apex is created by applying vacuum pressure through space 65 between inner tube 62 and outer tube 64 of cannula 60. Thus, a vacuum is formed between the suction cup, and the tissue of the heart surrounding the hole, thereby sealing the suction cup to the tissue that surrounds the hole in the apex. Typically, balloon 72 provides sealing of the cannula with respect to the inner surface of the wall of the heart at the apex. FIGS. 6B and 6C show respective views of cannula 60 at the stage when inner tube 62 has been slid distally with respect to outer tube 64, and balloon 72 has been inflated.

As described hereinabove, subsequent to sealing the tissue of the heart that surrounds the hole with respect to the suction cup and the securing of the wall of the heart between the balloon and the suction cup, the dilator is withdrawn from the inner tube of cannula 60. At this stage, inner tube 62 of cannula 60 provides a working channel from outside the subject's chest to inside the subject's heart, via the hole in the apex of the heart. The distal end of the cannula is sealed with respect to the tissue that surrounds the hole at the apex, as described hereinabove. For some applications, a further tube that is disposed within inner tube 62 of the cannula provides the working channel of the cannula. Cannula 60 typically provides a working channel from outside the subject's chest, through trocar 40, and into the subject's heart via the hole in the apex.

Typically, a cardiac interventional procedure is performed with respect to the subject's heart, using the working channel that has been created through the patient's chest into the subject's heart via the hole at the apex. Typically a working catheter that is used to perform the procedure is inserted into the subject's heart via the working channel. For example, a valve of the subject's heart may be repaired or replaced, or a different cardiac structure may be repaired or replaced. Alternatively or additionally, the working channel can provide access to the subject's heart to facilitate any other type of cardiac surgery that can be performed with a minimally-invasive approach, such as ablation of a heart wall, implantation of a cardiac assist device, repairing a structural defect of the heart, repair of a failed bioprosthesis, treatment of atrial fibrillation, and/or transvascular approach to repairing or implanting a device in the ascending aorta, the aortic arch, and or the carotid arteries. Accordingly, any suitable delivery system (e.g., any other catheter used in the art) may be used to penetrate the passage in the heart for delivery of the tool. For example, an inner tube (not shown), e.g., a Transcatheter Aortic-Valve Implantation (TAVI) introducer sheath, or an introducer sheath of a prosthetic mitral valve, may be advanced through cannula 60 for delivery of an element required for valve implantation.

Reference is now made to FIG. 6D, which is a schematic illustration of cannula 60 that is typically inserted through trocar 40 and through a hole in the apex of the subject's heart, in accordance with some applications of the present invention. It is noted that, for some applications, the cannula is inserted between the subject's ribs and through the hole in the subject's apex, in the absence of trocar 40.

For some applications, as described with reference to FIGS. 6A-C, balloon 72 is disposed on inner tube 62, the balloon being configured to be disposed within the heart chamber and to be pulled back against the inner surface of the wall of the heart. For some applications, balloon 72 is flared distally (e.g., the distal surface of the balloon is concave in the distal direction), so as to reduce the extent to which the balloon protrudes into the left ventricle. For some applications, the balloon has a proximally-facing-nipple shape (e.g., the proximal surface of the balloon may be generally convex in the proximal direction) so as to conform with the shape of the inner surface of the wall of the heart, thereby facilitating sealing between the balloon and the wall of the heart. Alternatively, the balloon has a different shape, e.g., a toroidal shape, a round shape, and/or an elliptical shape. For some applications, a second balloon 74 is disposed on the distal end of outer tube 64, the second balloon being configured to be disposed outside the heart and pushed against an outer surface of the wall of the heart. Typically, the second balloon is flared distally (e.g., a distal surface of the second balloon may be concave in the distal direction, and/or the distal surface of the second balloon may have a proximally-facing-nipple shape) so as to conform with the shape of the outer surface of the wall of the heart and to form a seal against the outer surface. Typically for such applications, cannula 60 defines a second inflation port 68 for inflating balloon 74.

Inner tube 62 and outer tube 64 of cannula 60 are typically movable with respect to one another, as described with reference to FIGS. 6A-C. For some applications, second balloon 74 is inflated and is pushed against the outer surface of the wall of the heart. Subsequently, the first balloon is inflated and pulled toward the second balloon by moving inner tube 62 proximally with respect to outer tube 64, thereby sandwiching the wall of the heart between the first and second balloons. Alternatively, the first balloon is inflated first and is pulled against the inner surface of the wall of the heart. Subsequently, the second balloon is inflated and is pushed toward the first balloon by pushing outer tube 64 distally with respect to inner tube 64, thereby sandwiching the wall of the heart between the first and second balloons. For some applications, space 65 between inner tube 62 and outer tube 64 is used as a suction lumen, to facilitate sealing of second balloon 74 against the outer surface of the wall of the heart.

For some applications, balloons 72 and 74 assume the shapes shown in FIGS. 6A-D when the balloons are in partially inflated states thereof. Alternatively or additionally, the balloons assume the shapes shown in FIGS. 6A-D when the balloons are in fully inflated shapes thereof.

For some applications, as an alternative to, or in addition to, using cannula 60, shown in FIGS. 6A-D, once a dilated path is created by a dilator, a tool (not shown), such as a tube, which is of the type that is sometimes referred to as a cannula, tube, port, or catheter (hereinafter, collectively referred to as “the catheter”) is inserted into the enlarged path over guidewire 10 or guidewire 15. For example, the catheter may be generally similar to catheter 62, described with reference to FIG. 5 of U.S. Provisional Patent Application 61/452,465, and/or with reference to FIGS. 5A-C of U.S. Provisional Patent Application 61/475,751, both of which applications are incorporated herein by reference. Following are descriptions of techniques that are practiced using such a catheter, in accordance with some applications of the present invention.

The catheter may be of circular or non-circular cross-section, and the cross-section may vary over the length of the catheter. Alternatively, the catheter itself serves as the dilator, and may be, for example, plastically deformable and radially enlarged by inflation of a balloon within the cannula. For some applications, a 20-40 Fr catheter, e.g., a 25-32 Fr catheter, is inserted over guidewire 10 or guidewire 15. The catheter typically has a distal end comprising, for example, a shape memory alloy material which is configured to expand within the subject's body into a flared distal end.

For some applications, a proximal portion of the catheter includes threading on an outer surface thereof. The catheter is advanced toward the left ventricle (e.g., over guidewire 10 or guidewire 15), by the proximal portion being screwed through an opening in the subject's skin. For some applications, advancing the catheter in this manner facilitates gradual advancement of the catheter. Gradual advancement of the catheter may reduce the likelihood of the subject's heart being injured by the distal tip of the catheter penetrating tissue of the subject's heart, relative to if the catheter were advanced in a less gradual manner. For some applications, the threading facilitates anchoring of the catheter to soft tissue in the vicinity of the opening in the subject's skin through which the catheter is inserted. For some applications, a proximal portion of another of the tools described herein is threaded in order to facilitate the gradual advancement of the tool toward the subject's heart.

For some applications, the flared distal end of the catheter comprises a plurality of flexible plate elements, which are slidably coupled to each other to form the final flared shape. Alternatively or additionally, the flared distal end comprises a stent structure, e.g., comprising nitinol and typically lined by a generally-impermeable membrane. The stent structure expands to a pre-trained flared shape upon removal of a tube surrounding the distal end of the catheter, in a similar manner to that described with reference to suction cup 77.

For some applications, the flared distal end of the catheter comprises rubber or another mechanically-similar material, which is folded or wrapped to fit into a tube, but which upon removal of the tube expands to form the flared shape of the flared distal end. For example, during advancement of the catheter toward the subject's heart, the flared distal end may be disposed inside the catheter. When the distal end of the catheter is disposed in the vicinity of the heart, the flared distal end is pushed out of the distal end of the catheter. Typically, the flared distal end defines at least one lumen therethrough. For example, the flared distal end may have a similar configuration to a laparoscopic port device, e.g., the SILS™ Port Multiple Instrument Access Port manufactured by Covidien (MA, USA).

Regardless of which of the above options is utilized to cause the desired flaring, the flared distal end may optionally be placed back into the tube prior to withdrawal from the chest cavity. It is noted that immediately outside of the apex, a natural anatomical space exists which facilitates the use of the flared distal end and the other techniques described herein which are practiced outside of the apex, such as the placement of suction cup 77 outside the apex. For some applications, such a space does not exist, e.g., when there has been a growth of post-operational adhesion tissue within the space. For some such applications, a balloon or a balloon-like device is typically inflated in the chest cavity immediately outside the apex, so as to create a space that facilitates the use of the flared distal end and the other techniques described herein which are practiced outside of the apex, such as the placement of suction cup 77 outside the apex. Alternatively, for some such applications, cannula 60 is sealed with respect to the apex via an intra-cardiac sealing element, e.g., by pulling balloon 72 proximally against the inner surface of the wall of the heart at the apex. For some applications, balloon 72 is pulled proximally against the inner surface of the wall of the heart at the apex, such as to pull the apex toward the subject's chest.

The flared distal end of the catheter typically facilitates contact of the catheter with the external side of the heart wall and typically provides sealing of the hole through the apex and aids in reducing leakage of blood from the hole. For example, the catheter may comprise a sealing element over its flared distal end to reduce blood leakage. Or, a flared distal end that is advanced out of the distal end of catheter may form a seal against the external side of the heart wall, as described hereinabove.

Additionally or alternatively, the catheter may have a valve coupled to its proximal end which enables suction functionality in order to clear internal blood leakage during the procedure. Further additionally or alternatively, the catheter may be a multi-lumen catheter having one or more lumens for applying suction to the distal end of the catheter to facilitate contact of the catheter (and/or the flared distal end of the catheter) with the external side of the heart wall and/or to remove blood from the site of the passage. One or more additional lumens of the catheter may be used for passage of tools, e.g., sutures and needles, therethrough.

Typically, the catheter provides an enlarged path extending from the skin to the heart, which is used for passage of tools into the heart. As described herein, the tools may comprise any element required for valve treatment or replacement, e.g., a prosthetic valve. Additionally or alternatively, the tool may comprise any tool required for any cardiac procedure, e.g., an ablation tool for ablation of a site in the wall of the heart. Accordingly, any other suitable delivery system (e.g., any other catheter used in the art) may be used to penetrate the passage in the heart for delivery of the tool. For example, an inner tube (not shown), e.g., a Transcatheter Aortic-Valve Implantation (TAVI) introducer sheath, or an introducer sheath of a prosthetic mitral valve, is advanced through the catheter for delivery of an element required for valve implantation.

For some applications, a single catheter generates the passage through the tissue extending from the skin to the heart, and is then advanced through the subject's myocardium and into the subject's left ventricle, thereby providing a passage from the subject's skin, into the subject's heart. Alternatively, a first catheter generates the passage through the tissue extending from the skin to the heart, but is not advanced through the subject's myocardium and into the subject's left ventricle. Rather, a second catheter is advanced through the passage through the tissue from the skin to the heart that was generated by the first catheter (e.g., by the second catheter being advanced through the first catheter), and the second catheter is then advanced through subject's myocardium and into the subject's left ventricle. For some applications, the first and second catheter comprise inner and outer tubes of a single catheter or cannula, e.g., as described hereinabove with reference to cannula 60. For some applications, subsequent to the second catheter being advanced into the subject's heart, the first catheter is removed from the subject's body. For some applications, the first catheter is removed from the subject's body, and the flared distal end of the first catheter remains adjacent to the heart wall, the second catheter passing through a port in the flared distal end and into the subject's heart. For some applications, subsequent to the second catheter having been placed so as to provide a passage from the subject's skin into the subject's heart, a further tube, e.g., a Transcatheter Aortic-Valve Implantation (TAVI) introducer sheath, or an introducer sheath of a prosthetic mitral valve, is advanced through the second catheter and into the subject's heart.

For some applications, the first catheter generates the passage through the tissue extending from the skin to the heart and the flared distal end of the first catheter is placed against the wall of the heart. Subsequently, the first catheter is removed, and the flared distal end remains adjacent to the heart wall, the flared distal end defining a port that provides a passage from the subject's skin to the subject's heart. Thus, subsequent to the removal of the first catheter, tools are inserted from the subject's skin into the subject's heart via the port that is defined by the flared distal end.

In accordance with some applications of the present invention, the path from the skin to the heart created by guidewire 10 or guidewire 15 is dilated, e.g., as described hereinabove. Dilation of the path typically facilitates passage of large tools into the chamber of the heart, thus preventing the need for an incision e.g., a thoracatomy, used in transapical transcatheter procedures known in the art.

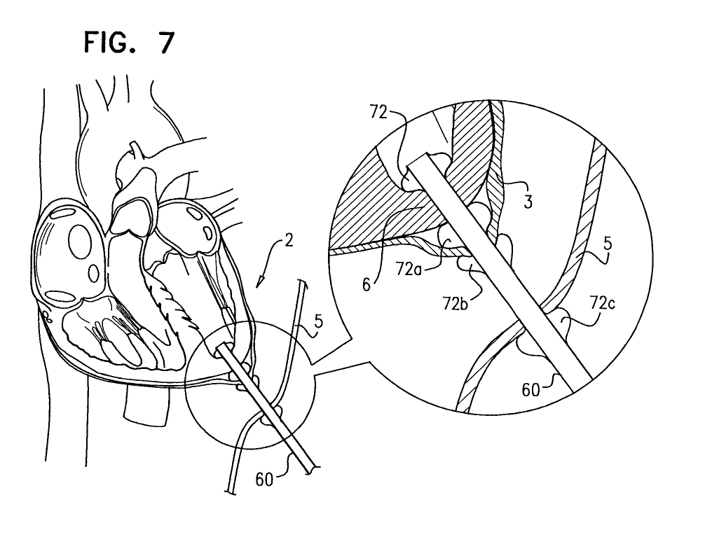

Reference is now made to FIG. 7, which is a schematic illustration of cannula 60, the cannula having a plurality of balloons 72, 72a, 72b, 72c, disposed thereon, in accordance with some applications of the present invention. In other aspects, cannula 60 is generally similar to cannula 60 described hereinabove with respect to FIGS. 6A-D. Typically, the balloons act as space-occupying elements. For some applications, an element other than a balloon (e.g., an expandable structure other than a balloon) is used as a space-occupying element instead of one or more of the balloons, mutatis mutandis. For some applications, one or more space-occupying elements (e.g., balloons, as shown) are coupled to or near the distal tip of a cannula (such as cannula 60, as shown) which was introduced from the skin through the chest wall into the heart, and is placed inside a chamber of the heart, e.g., inside the left ventricle. Typically, a device, such as a Transcatheter Aortic-Valve Implantation (TAVI) introducer sheath, or an introducer sheath of a prosthetic mitral valve, is inserted into the heart via the cannula having the space-occupying elements coupled to or near its distal tip.

The space-occupying elements may comprise any suitable three-dimensional structure e.g., a stent or a multiple wire configuration or an inflatable element. As shown in FIG. 7, the space-occupying elements comprise multiple balloons 72, 72a, 72b, 72c, which are coupled to or near the distal tip of cannula 60, which was introduced from the skin through the chest wall into the heart, and placed inside the left ventricle. It is noted that any suitable number of balloons may be used, and that, as appropriate, one or more of the balloons may be biodegradable. One or more of the balloons is typically inflated in the heart chamber. The proximal end of the cannula is manipulated by the surgeon. For some applications, the surgeon may pull the proximal end of the cannula in a direction that is away from the subject's body, thus pulling the one or more balloons against the wall of the heart and thereby pulling the heart closer to the chest wall.

Inflation of one or more of the balloons against the wall of the ventricle typically additionally provides sealing of the puncture site by the balloon, and aids in reducing leakage of blood from the puncture site. Alternatively or additionally, the balloons coupled to the cannula inhibit the cannula from inadvertently slipping out the heart. Further alternatively or additionally, the balloons coupled to the cannula provide stabilization while tools are introduced, thereby helping to reduce bleeding.

The balloons may comprise materials configured to be stiff or compliant, such as nylon, silicone, latex or polyurethane. For some applications, the balloons comprise a mixture of materials for providing a balloon of varying stiffness, such that one side of the balloon is compliant (e.g., the heart-facing side), while the other side is stiffer.

The balloons may in principle have any suitable shape (e.g., spherical, ellipsoidal, toroidal, hourglass, or cylindrical). For some applications, the maximum length of one or more of the inflated balloons, measured along the longitudinal axis of the cannula, is smaller than the maximum length of the inflated balloon measured perpendicular to the longitudinal axis of the cannula. For example, the inflated balloon may be disk-shaped, and one side of the disk may be pressed against the interior wall of the heart chamber in which the balloon is disposed. In this manner, for a relatively low inflation volume of the balloon, a relatively large area of the external surface of the balloon is provided for applying force to the interior wall of the heart chamber. For some applications, the maximum length measured perpendicular to the longitudinal axis of the cannula is at least 30 percent (e.g., at least 100%) greater than the maximum length of the inflated balloon measured along the longitudinal axis of the cannula.

For some applications, the cannula may have multiple balloons 72, or any other space-occupying elements that function as inflatable and/or sealing elements, that may be positioned at any anatomical layer along the path created between the heart and the skin of the chest wall, or outside of the chest wall (e.g., on the skin). For example, the balloons may be positioned between the myocardium and the pericardium 3, and/or between the pericardium and the chest wall, or in any other location along the aforementioned path. FIG. 7 shows a cannula with multiple balloons: a first balloon 72 positioned within the left ventricle against the wall of heart 2 (generally similar to balloon 72, as described hereinabove), a second balloon 72a placed between the wall of heart 2 (myocardium) and pericardium 3, a third balloon 72b placed between pericardium 3 and chest wall 5, and a fourth balloon 72c placed outside of the chest wall. The balloons typically lock the anatomical structures and the cannula together, in order to reduce relative motion between any of the anatomical structures and the cannula.

For some applications; one or more of the balloons shown in FIG. 7 may be coupled to an additional element, e.g., an electrode or a cutting device. For example, one or more balloons (e.g., balloon 72a) placed between the wall of heart 2 (myocardium) and pericardium 3, may be coupled to a sharp element and/or an electrode configured for cutting an incision in pericardium 3 in order to allow passage of fluid therethrough (e.g., a pericardial window), thus preventing accumulation of blood or fluid between myocardium and the pericardium which may lead to pericardial tamponade. For such an application, a scoring balloon may be used, e.g., placed inside or outside cannula 60. The scoring balloon is typically passed through cannula 60 in order to arrive at a location where it is used to create the incision in the pericardium. Alternatively, a balloon coupled to a sharp element and/or an electrode may be placed between the myocardium and the pericardium (application not shown) for creating an incision in the pericardium.

For some applications, instead of or in addition to the electrode or cutting device disposed on a balloon as described hereinabove, a catheter is passed through a hole in a lateral wall of cannula 60, and an electrode or cutting device is passed through the catheter and is used to create an opening in the pericardium, e.g., to prevent tamponade. Alternatively or additionally, the catheter is passed through an incision in the skin that is separate from the incision through which cannula 60 is passed, and the electrode or cutting device is passed through the catheter and creates the opening in the pericardium.

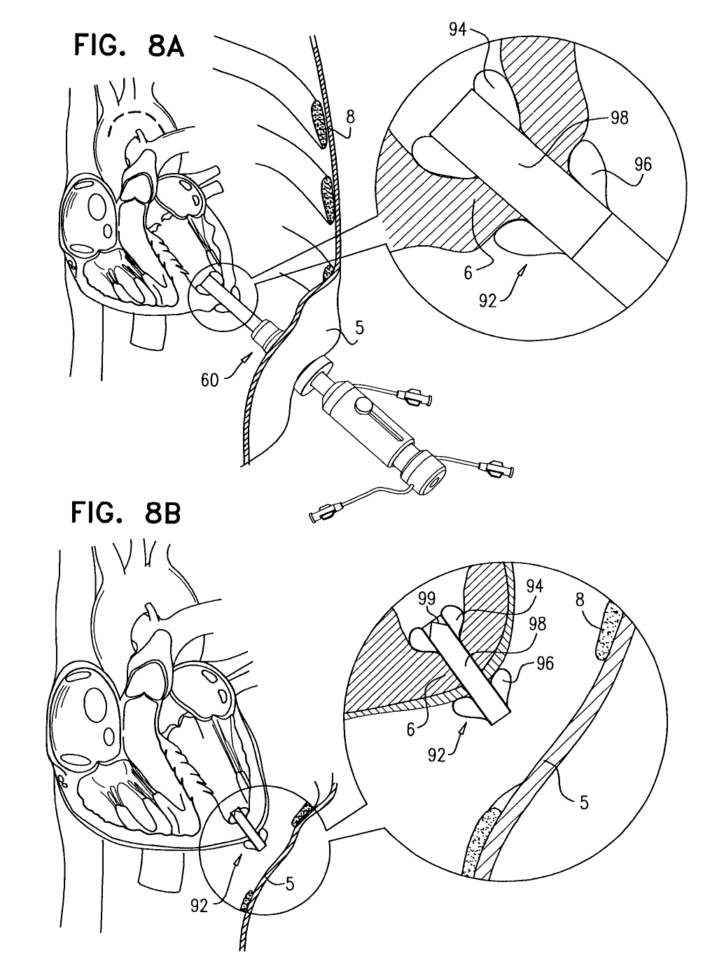

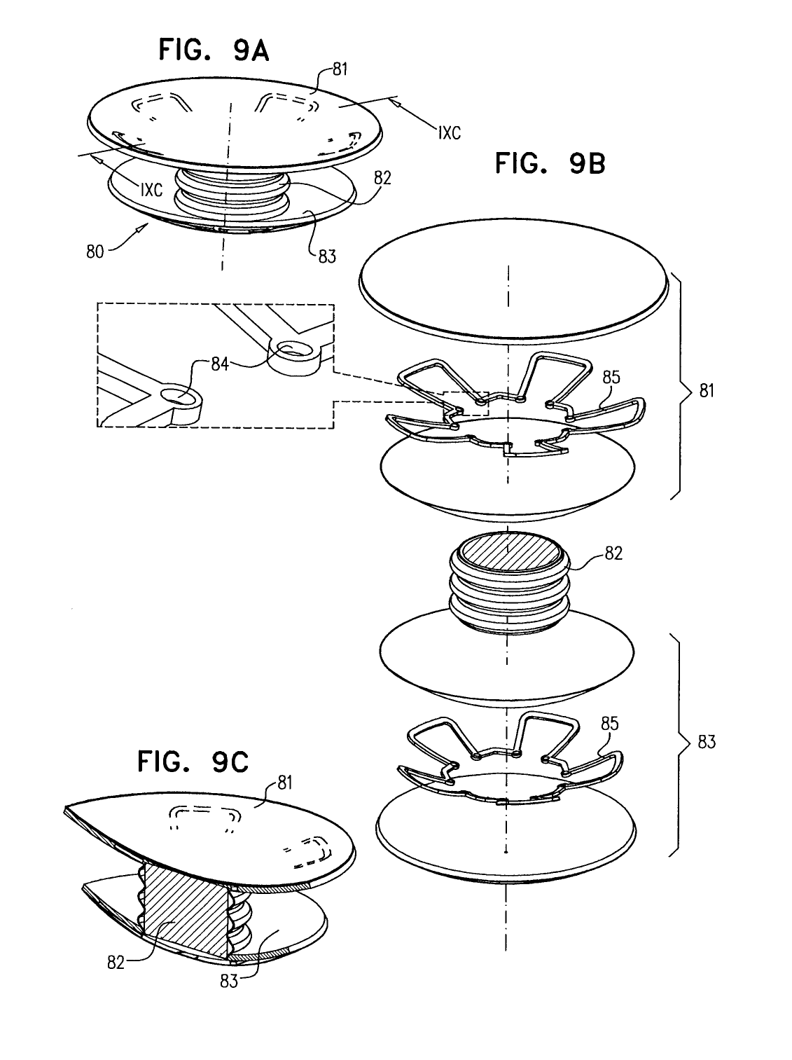

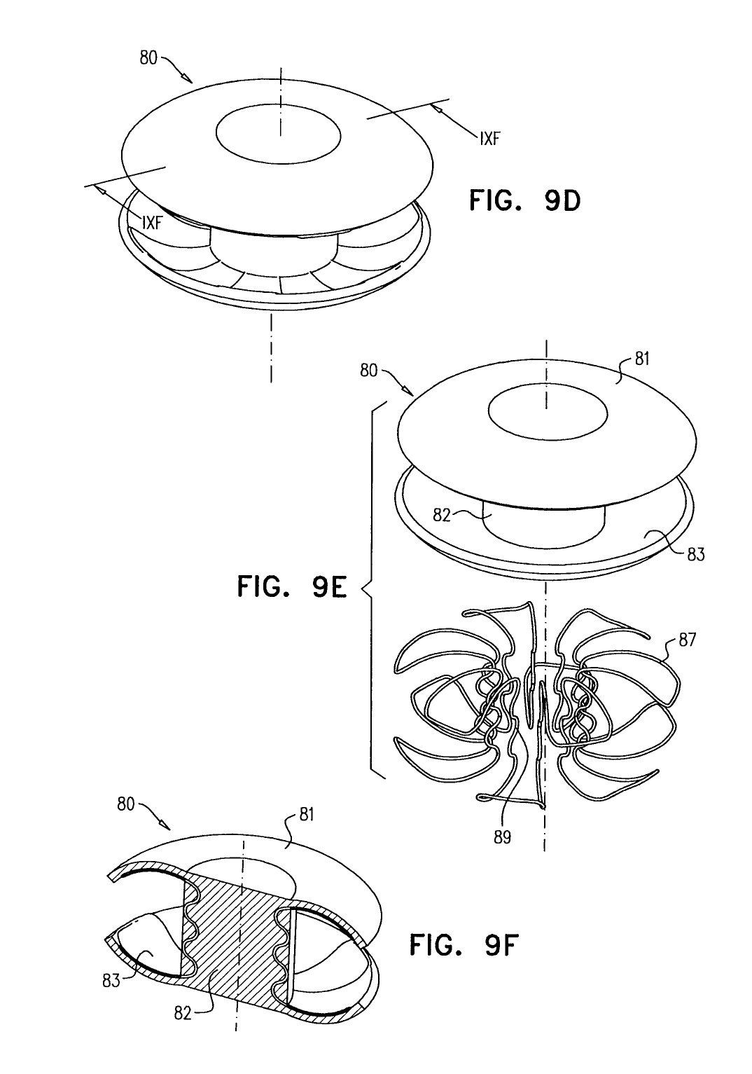

Reference is now made to FIGS. 8A-B, which are schematic illustrations of a closure device 92 for sealing a passage in the heart, the device being configured to be disposed around a distal portion of cannula 60 as an overtube, in accordance with some applications of the present invention. It is noted that, although device 92 is shown as being configured as an overtube for being placed around the distal portion of the cannula, for some applications, device 92 is coupled to the cannula in a different manner, e.g., by configured as an extension from the distal end of the cannula, such as by being coupled to the distal end of the cannula via a coupling mechanism. For some applications, closure device 92 includes first and second balloons 94 and 96, which are disposed on a support member 98. The support member is configured such that it can be reversibly placed on a distal portion of cannula 60, and/or a different cannula and/or catheter that is inserted through the subject's skin into the subject's heart. At least one hemostatic valve 99 (shown in FIG. 8B) is typically disposed inside the support member. The valve is configured such that when the support member is placed on the cannula (as shown in FIG. 8A), the valve is opened, and when the cannula is removed from inside the support member (as shown in FIG. 8B), the valve closes, so as to form a hemostatic seal. Alternatively or additionally, the lumen of support member 98 is closed and/or sealed using a plug, such as a sponge or a foam plug. For example, the plug may be generally similar to closure device 80 described hereinbelow with reference to FIGS. 9A-F.

For some applications, cannula 60 (or a different cannula or catheter) is advanced into the subject's heart (as described hereinabove), support member 98 being disposed on a distal portion of the catheter, and the balloons being in deflated states (not shown). When first balloon 94 is disposed within the heart chamber and second balloon 96 is placed between the pericardium and the chest wall, the balloons are inflated with an inflation fluid (e.g., saline), as shown in FIG. 8A. (For some applications, the first balloon is inflated first and the balloon is pulled back against the inner surface of the heart wall, and subsequently, the second balloon is inflated.) Subsequent to a procedure having been performed via cannula 60, e.g., as described hereinabove, the cannula is removed from the subject's body, leaving balloons 94 and 96 and support member 98 in place. For some applications, removal of the catheter causes hemostatic valve 99 to close, thereby forming a hemostatic seal. Alternatively or additionally, a plug is inserted into the lumen of support member 98 subsequent to the removal of the catheter therefrom, as described hereinabove. Balloons 94 and 96, support member 98, and hemostatic valve 99 facilitate sealing of the hole in the heart wall, subsequent to the removal of the cannula from inside support member 98.

For some application, when balloons 94 and 96 have been suitably positioned and inflated so as to seal the hole in the wall of the heart, a thermosetting material, such as epoxy, is injected into one or both of the balloons, via injection lumens (not shown). The inflation fluid (e.g., the saline) that was used to inflate the balloons, is removed from one or both of the balloons via drainage lumens (not shown). The thermosetting material hardens (i.e., changes from a fluid state to a solid state thereof) inside the balloons, thereby maintaining the shape of the balloons.

Reference is now made to FIGS. 9A-F, which are schematic illustrations of closure devices 80 for sealing the passage in the heart, in accordance with some applications of the present invention. Reference is also made to FIGS. 10A-D, which are schematic illustrations of a procedure for sealing a hole in the wall of a subject's heart using closure device 80, in accordance with some applications of the present invention.

For some applications, closure device 80 is passed into the heart over the guidewire 10 or 15, and/or through a cannula (e.g., cannula 60) or a catheter, through a path that was created from the heart to the chest wall, for example, using the techniques described herein. As shown in FIG. 10A, typically, the closure device is advanced through the working channel of cannula 60 (e.g., through inner tube 62 of cannula 60). For some applications, the Transcatheter Aortic-Valve Implantation (TAVI) introducer sheath which was used for delivery of tools for valve repair, or an introducer sheath of a prosthetic mitral valve, is used for delivery of closure devices to the heart. Generally, the closure device may be advanced to the hole in the apex of the heart in a compressed and/or folded state thereof, and may expand into an operable state upon insertion into a cardiac structure. The closure device may be introduced either through the path established from the skin into the heart, or from within the heart outwards toward the skin, or a combination of these two paths. Advantageously, some procedures described herein provide a surgeon with access to the heart from both inside the heart and from outside the heart.

As shown in FIGS. 9A-F, for some applications, the closure device 80 comprises a plug portion 82 configured for placement within the passage in the heart wall, an intracardiac portion 81, coupled to the plug portion, and configured for placement within the heart chamber, and an extracardiac portion 83 coupled to the plug portion and configured for placement outside the heart chamber. Although plug portion 82 is shown in FIGS. 9A-C as having a being shaped to define protrusions on its outer surface, for some applications, plug portion 82 has a smooth outer surface.

Plug portion 82 typically comprises a transmural plug portion comprising a biodegradable and/or bioabsorbable and/or degradable implantable material and/or a cloth and/or a sponge, e.g., surgical cloth. The transmural plug portion is configured for placement within the passage in the wall of the heart, and is configured to conform, e.g., expand, to the size and shape of the passage, such that the plug provides sufficient sealing of the passage by occupying the entire space of the passage. For some applications, the biodegradable plug comprises a material such as PGA and/or collagen. For some applications, the plug is not biodegradable. For some applications, the plug comprises a nitinol and/or a stainless steel and/or a cobalt chromium structure. The plug may comprise any other suitable material, e.g., plastic and/or nylon.

Typically, plug portion 82 comprises an expansible material. The plug portion is configured to be placed at the hole via an insertion device, such as cannula 60, while the plug portion is constrained by the insertion device. Upon being pushed out of the insertion device, the plug portion is configured to expand radially since the plug portion is no longer constrained by the insertion device. Thus, the plug portion expands to fill the hole in the apex. For example, if the radius of the hole is 5 mm (e.g., due to cannula 60 having an outer radius of 5 mm), then during insertion of the plug portion, the plug portion may be maintained in a radially-compressed state due to being constrained by the cannula, the plug portion defining an outer radius of 4 mm in the radially-compressed state. Upon being pushed out of the cannula, the plug portion radially expands to a radius of at least 5 mm, thereby sealing the hole. Typically, the plug portion is configured to expand radially by more than 0.5 percent, e.g., more than 5 percent between the constrained state of the plug portion (inside the insertion device) and the unconstrained state of the plug portion, even in the absence of any radial expansion of the plug portion that is due to absorbance of fluid by the plug portion. For example, the plug portion may be configured to radially expand by 0.5-100 percent, e.g., 0.5-5 percent, and/or 5-100 percent between the constrained state of the plug portion (inside the insertion device) and the unconstrained state of the plug portion.

Alternatively or additionally, plug portion 82 comprises an absorbent material, the plug portion thereby being configured to expand radially due to absorbing body fluids, i.e., it swells in the presence of fluid. Typically, the plug portion is configured to expand radially by more than 0.5 percent, e.g., more than 5 percent due to absorbing fluids. For example, the plug portion may be configured to radially expand by 0.5-100 percent, e.g., 0.5-5 percent, and/or 5-100 percent due to absorbing fluids. For some applications, the radial expansion of the plug portion due to the absorbance of fluid by the plug portion, is in addition to the radial expansion of the plug due to expansible properties of the plug portion itself, as described hereinabove.

Thus, the plug portion facilitates sealing of the hole at least partially by radially expanding such that the outer surface of the plug portion comes into contact with the inner surface of the wall of the heart that defines the hole at the apex. For some applications, the radial expansion of the plug is accompanied by longitudinal shortening of the plug, e.g., 0.5-50 percent shortening (for example, 0.5-5 percent, or 5-50 percent). Alternatively, the plug does not shorten longitudinally. These values are typically observed in the absence of any forces applied to the plug (e.g., if the plug were not implanted in the heart). Some changes in the values may be expected based on the properties of any individual subject's heart, and the nature of the passage into which the plug is placed.

Typically, intracardiac portion 81 of closure device 80 is coupled to plug portion 82, and is configured for placement within the heart chamber. Typically, the intracardiac portion becomes coupled to the cardiac wall in a vicinity of the passage and facilitates anchoring of the plug portion within the passage. For some applications, intracardiac portion 81 of closure device 80 generally conforms to the shape of the inner cardiac wall. For example, in accordance with some applications of the present invention, the passage is created in the apex of the left ventricle. For such applications, the intracardiac portion typically defines a conical shape (e.g., an upwardly-concave (i.e., concave in the distal direction) disc shape, as shown) fitting into the apex inside the left ventricle. Alternatively, the intracardiac portion may be shaped to define any other shape that facilitates anchoring of the plug portion within the passage, e.g., a torus, a disc shape, or a mesh, coupled to the plug. The intracardiac portion of the closure device typically comprises nitinol or stainless steel (e.g., a nitinol or stainless steel mesh, and/or nitinol or stainless steel struts), which materials may facilitate tissue growth (e.g., growth of endothelial tissue) on the surface of the intracardiac portion and reduce any chronic adverse immune reaction. For some applications, the intracardiac portion includes a fabric, such as a polyethylene terephthalate cloth, and/or any other material that may be used as an impermeable patch.

Typically, closure device 80 further comprises extracardiac portion 83 that is coupled to plug portion 82 and configured for placement outside the heart chamber. Typically, the extracardiac portion becomes coupled to an external side of the cardiac wall in a vicinity of the passage and facilitates anchoring of the plug portion within the passage. For some applications, the extracardiac portion of the closure device conforms to the shape of the outer cardiac wall. For example, in accordance with some applications of the present invention, the passage is created in the apex of the left ventricle, and the extracardiac portion defines an upwardly-concave cap-shape or disc-shape fitting onto the apex from outside the left ventricle.

As described hereinabove, for some applications, intracardiac portion 81 and extracardiac portion 83 define upwardly-concave disc shapes. Typically, the intracardiac portion defines an upwardly-concave disc shape having a radius of curvature that is greater that the radius of curvature of the intracardiac side of the apex, so as to facilitate sealing of the intracardiac portion of the closure device with respect to the intracardiac side of the apex. Further typically, the extracardiac portion defines an upwardly-concave disc shape having a radius of curvature that is less that the radius of curvature of the extracardiac side of the apex, so as to facilitate sealing of the extracardiac portion of the closure device with respect to the extracardiac side of the apex. Therefore, typically, intracardiac portion 81 of closure device 80 has a greater radius of curvature than does extracardiac portion 83. In alternative applications, intracardiac portion 81 of closure device 80 has a smaller radius of curvature than does extracardiac portion 83.

Typically, intracardiac portion 81, plug portion 82, and extracardiac portion 83 of hole closure device 80 are movable with respect to each other such that the portions can conform to anatomical variations and asymmetry of the subject's heart. Further typically, intracardiac portion 81, plug portion 82, and extracardiac portion 83 of hole closure device 80 are movable with respect to each other such that the portions can maintain a seal around the hole in the heart, even when the heart moves, by portions 81, 82 and 83 moving with respect to each other, so as to conform to movement of the subject's heart.

As shown in FIGS. 9A-C, for some applications, intracardiac portion 81 and/or extracardiac portion 83 of closure device 80 include struts 85. For example, the struts may be formed of a shape-memory material, such as a shape memory alloy (e.g., nitinol). Typically, upper and lower layers of fabric cover the struts of the intracardiac portion and upper and lower layers of fabric cover the struts of the extracardiac portion, as shown. Typically, the fabric layers are sutured to the struts using suturing holes 84. During insertion of the closure device into the hole in the subject's heart, the intracardiac and extracardiac portions are typically folded and radially compressed so as to facilitate insertion of the hole closure device through cannula 60, as described with reference to FIGS. 10A-D. For some applications, closure device 80 is configured such that when the closure device is in a non-constrained state, struts 85 cause intracardiac portion 81 and extracardiac portion 83 to have upwardly-concave disc shapes, e.g., as described hereinabove. For some applications, the struts that are disposed respectively in the intracardiac and the extracardiac portions do not define a single, integral structure. Rather, separate strut structures are disposed respectively in the intracardiac and the extracardiac portions. For some applications, plug portion 82 includes an absorbent material as described hereinabove, and does not include any rigid materials, such as a rigid frame configured to impart rigidity to the plug portion.

As shown in FIGS. 9D-F, for some applications, a single integral frame 87 is disposed inside closure device 80. For example, the frame may be formed of a shape-memory material, such as a shape memory alloy (e.g., nitinol). For some applications (not shown), closure device 80 is configured such that when the closure device is in a non-constrained state, frame 87 causes intracardiac portion 81 and extracardiac portion 83 to have upwardly-concave disc shapes, e.g., as described hereinabove. For some applications (as shown), frame 87 causes intracardiac portion 81 to have an upwardly-convex (i.e., convex in the distal direction) disc shape, and extracardiac portion 83 to have an upwardly-concave disc shape.

For some applications, a central portion 89 of frame 87 is disposed inside plug portion 82 of the closure device. For example, the central portion of the frame may impart rigidity to the plug portion. Alternatively or additionally, the central portion of the frame may be configured to cause the plug portion to radially expand when the plug portion is in a non-constrained state. It is noted that even for applications in which a frame is disposed inside plug portion 82, nevertheless more than 50 percent of the non-constrained volume of the plug portion comprises an expansible material, as described hereinabove. Alternatively, more than 50 percent of the non-constrained volume of the plug portion comprises an arrangement of materials, such that the arrangement is expansible, even if the materials themselves are not substantially expansible. Furthermore, even for applications in which a frame is disposed inside plug portion 82, nevertheless, at least the outer layer of the plug portion, which comes into contact with the wall of the heart that defines the hole at the apex of the heart, typically includes a soft, absorbent material. Typically, having a plug portion having a soft outer layer reduces damage caused to myocardial tissue surrounding the hole in the heart by the hole closure, relative to a hole closure device that has a rigid (or partially rigid) outer layer thereof.

For some applications, frame 87 is pre-shaped such that the frame tends to shorten plug portion 82, when the wire structure is unconstrained. Typically, the longitudinal compression of the plug portion compresses tissue of the wall of the heart in the vicinity of the closure device thereby sealing the wall of the heart against the closure device. For some applications, the shortening of the plug portion causes the plug portion to expand radially. For some applications, plug portion 82 of the closure device is configured to expand radially even if the plug portion does not become longitudinally compressed. For some applications, frame 87 is pre-shaped so as to cause plug portion to expand radially, when the plug portion is not radially constrained by the catheter. Typically, the plug portion is made from an expansible material (e.g., a sponge). The plug portion is compressed when the plug portion is within the catheter and expands radially upon protruding from the catheter. Typically, the radial expansion of the plug portion seals the plug portion against the opening in the wall of the heart.

It is noted that closure device 80, as shown in FIGS. 9A-C and as shown in FIGS. 9D-F, does not include any rigid materials across substantially the entire diameter (e.g., more than 90 percent of the diameter) of the plug portion. For some applications, the plug portion is thus configured to facilitate insertion of a medical tool (such as a catheter) through the apex of the heart, by the tool being inserted through the plug portion. The plug portion is typically further configured to automatically seal the hole in the apex subsequent to the removal of the medical tool from the plug portion, by the plug portion expanding.

Reference is again made to FIGS. 10A-D. As described hereinabove, subsequent to performing a cardiac interventional procedure via the working channel (e.g., inner tube 62) of cannula 60, the tools that were used to perform the procedure are withdrawn from the working channel. Typically, subsequent to this step, closure device is placed inside the hole in the apex of the subject's heart in order to facilitate closure of the hole. As shown in FIG. 10A, closure device 80 is typically advanced through the working channel (e.g., inner tube 62) of cannula 60. For example, as shown, a pushing element 86 pushes the closure device distally through the working channel. Typically, while the closure device is advanced through the cannula, the hole-closure device is constrained by the cannula. For example, as shown, intracardiac portion may be folded into a distally-facing cup shape, and extracardiac portion 83 may be folded into a proximally-facing cup shape. For some applications, plug portion 82 is radially compressed during the advancement of the hole closure device through cannula 60.

Intracardiac portion 81 of the closure device 80 is deployed in the heart chamber by pushing the intracardiac portion out of the distal end of the working channel of cannula. As described hereinabove, the intracardiac portion is typically configured to automatically assume a shape that conforms with the inner surface of the wall of the heart (such as an upwardly concave disc-shaped shape) when the intracardiac portion is in a non-constrained state. Thus, the intracardiac portion assumes the shape, when the intracardiac portion is pushed out of the distal end of the cannula into the subject's heart, as shown in FIG. 10B.

Typically subsequent to the placement of intracardiac portion 81 of closure device 80 into the subject's heart, balloon 72, which is typically disposed at the distal end of inner tube of cannula 60, is deflated. The inner tube is typically then pulled proximally, thus pulling intracardiac portion 81 of the closure device against the inner surface of the wall of the heart, thereby placing the intracardiac portion in contact with the inner surface, as shown in FIG. 10C. Subsequently, the inner tube of the cannula is further retracted, such as to release plug portion 82 of the hole closure device from the inner tube of the cannula. For example, pushing element 86 may be configured to hold the hole-closure device stationary with respect to the subject's heart, while the inner tube of the cannula is retracted. The plug portion of the hole closure device is configured to automatically expand, such as to fill, and thereby form a plug, within the hole in the apex, as described hereinabove. Further subsequently, suction of suction cup 77 is terminated and outer tube 64 and inner tube 62 of cannula 60 are retracted from the subject's heart and out of the subject's chest through trocar 40. The retraction of the cannula is such as to cause extracardiac portion 83 of hole-closure device 80 to be released from inner tube 62. For example, pushing element 86 may be configured to hold the hole-closure device stationary with respect to the subject's heart while the cannula is retracted. As described hereinabove, the extracardiac portion is typically configured to automatically assume a shape that conforms with the outer surface of the wall of the heart (such as an upwardly concave disc-shaped shape) when the extracardiac portion is in a non-constrained state. Thus, the extracardiac portion assumes the shape, when the extracardiac portion is released from inner tube 62, as shown in FIG. 10D.

Closure device 80 is typically deployed such that extracardiac portion 83 of the device is deployed outside the pericardium. For some applications, the extracardiac portion is deployed between the myocardium and the pericardium. For some application, a portion of the pericardium is excised, and the extracardiac portion is deployed outside the myocardium.

For some applications, intracardiac portion 81 and extracardiac portion 83 are connected to the plug by a connecting element (not shown), e.g., a metal or polymeric wire that surrounds the plug, and pulling of the metal wire results in pulling of the intracardiac portion and the extracardiac portion towards each other, causing the plug to expand within the passage, thereby improving sealing of the passage (application not shown).

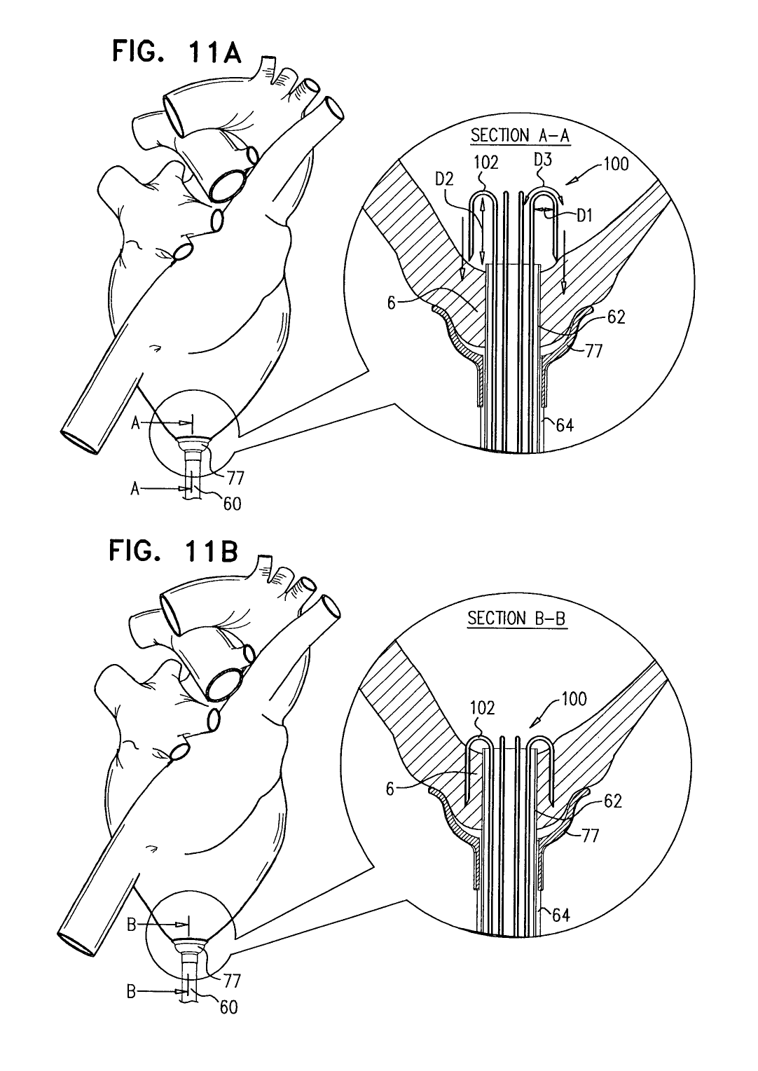

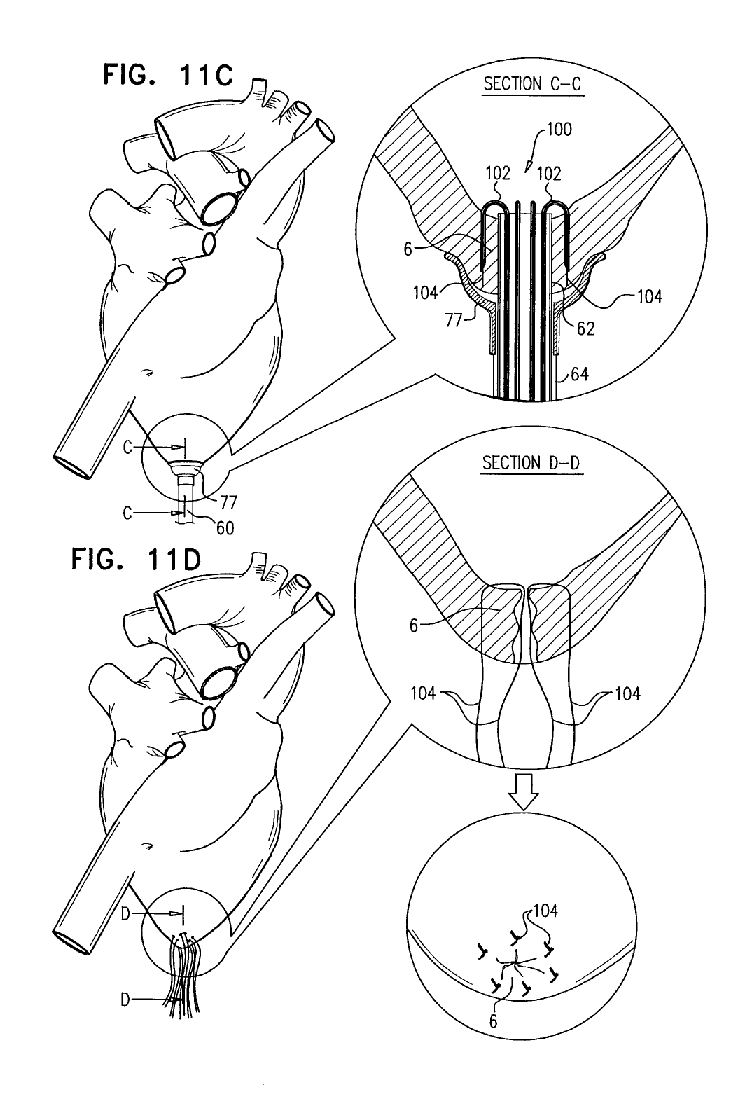

For some applications, additional anchoring mechanisms may be used in combination with the closure device in order to maintain the closure device in place. For example, a suturing system 100 described with reference to FIGS. 11A-D may be used in combination with the closure device. Optionally, a biodegradable suture is sutured through the plug portion, and extended out to the skin. The suture typically facilitates anchoring of the plug portion within the passage to prevent dislodging of the plug into the heart. Eventually, the biodegradable suture is dissolved into the body. Any other suitable anchoring options may be used as well. For some applications, pushing element 86 is maintained in contact with the closure device for a period of time subsequent to the placement of the closure device at the apex, and applies pressure to portions of closure device 80, in order to ensure proper positing of the plug and to secure the plug in place. Element 86 may be removed any time following the closure procedure through drainage tubes which typically remain in a subject following surgical procedures. For some applications, pushing element 86 is configured to temporarily seal the passage in the heart wall until closure device 80 is properly situated.

For some applications, hole-closure device is configured as described with reference to FIGS. 13E-J of U.S. Provisional Patent Application 61/475,751, which is incorporated herein by reference. For example, for some applications, closure device 80 is shaped to define plug portion 82 and intracardiac portion 81, the intracardiac portion having a greater cross-sectional area than the plug portion (when the plug and intracardiac portions are in non-constrained states thereof), but the closure device does not include an extracardiac portion having a greater cross-sectional area than the plug portion (application not shown). For some applications, closure device 80 is shaped to define plug portion 82 and extracardiac portion 83 that has a greater cross-sectional area than the plug portion (when the plug and extracardiac portions are in non-constrained states thereof), but the closure device does not include an intracardiac portion having a greater cross-sectional area than the plug portion (application not shown). Alternatively, the closure device includes intracardiac portion 81 and extracardiac portion 83, each of which has a greater cross-sectional area than the plug portion (when the plug, intracardiac, and extracardiac portions are in non-constrained states thereof), as shown in FIGS. 9A-F and 10A-D, for example. For some applications, the intracardiac portion and extracardiac portions have cross-sectional areas that are equal to one another. Alternatively, the cross-sectional of the intracardiac portion is greater than that of the extracardiac portion, or vice versa.

For some applications, closure device 80, or a portion thereof (e.g., plug portion 82) is configured to absorb blood, and includes coagulation-facilitating elements (not shown) that are configured to facilitate coagulation of the blood inside the closure device. For some applications, the coagulation-facilitating elements are coiled metallic elements, and/or other coagulation-facilitating elements that are known in the art. Alternatively or additionally, a surface of the closure device (e.g., a surface of intracardiac portion 81 of the device), and/or a portion of the device, is coated with a coagulation-facilitating coating, such as fibrin, and/or is covered with a material that contains fibrin. For some applications, the entire closure device is coated with a coagulation-facilitating coating, such as fibrin, and/or is covered with a material that contains fibrin.

For some applications, closure device 80 includes portions that comprise a shape-memory material, such as nitinol. For some applications, one or more tissue-coupling elements (e.g., pins, not shown) are disposed on intracardiac portion 81 and/or extracardiac portion 83 of the closure device. The tissue-coupling elements are pre-shaped, such that when the closure device is positioned within the wall of the heart, the tissue-coupling elements couple the closure device to the wall of the heart by becoming embedded in tissue of the wall of the heart.

For some applications, closure device 80 defines one or more channels therethrough (or through a portion thereof, application not shown). The closure device is configured such that, upon placement of the closure device within the wall of the heart, blood flows through the channels at a low flow rate. The slow blood flow through the channels facilitates coagulation of the blood within the channels, e.g., by causing stagnation flow thrombosis, thereby sealing the closure device. For some applications, a closure device that defines channels therethrough is used, the closure device or a portion thereof being made of a reticulated elastomeric material, and/or a reticulated foam that comprises polyurethane, polycarbonate polyurethane-urea, and/or a similar material.

It is noted that the scope of the present invention includes using the closure devices described herein (e.g., one or more of the devices described with reference to FIGS. 9A-F, 10A-D, and/or 11A-D) to close a structural heart defect, such as a ventricular septal defect, an atrial septal defect, and/or another structural heart defect.

For some applications, a hole closure device is used that is generally similar to that described with reference to FIGS. 15 and 16 of U.S. Provisional Patent Application 61/452,465, and/or with reference to FIGS. 15 and 16 of U.S. Provisional Patent Application 61/475,751, both of which applications are incorporated herein by reference, mutatis mutandis. For example, a closure device, e.g., a flexible ring, may be placed around inner tube 62 of cannula 60, distal to and contacting the distal end of a pushing tube. For some applications, the ring is pushed off of the inner tube by the pushing tube, placing anchoring elements of the closure device in the heart tissue. In order to protect tissue, for some applications, a pledget is attached to the anchoring elements. The ring typically automatically inverts to some extent after being pushed off inner tube 62, whereby the anchoring elements rotate to face each other, thereby closing the passage in the heart.

For some applications, a hole closure device is used that is generally similar to that described with reference to FIGS. 17A-B of U.S. Provisional Patent Application 61/452,465, and/or with reference to FIGS. 17A-B of U.S. Provisional Patent Application 61/475,751, both of which applications are incorporated herein by reference, mutatis mutandis. For some applications, a closure device for sealing the passage in the heart comprises an extracardiac clip (not shown) comprising a base portion coupled to two or more arms (e.g., three or four arms) and configured to engage the heart wall from the external side of the heart and facilitate closure of the passage in the heart. Typically, the arms of the clip surround the passage in the heart wall and contract, such that the punctured tissue of the heart wall is brought together, thereby facilitating closure of the passage. For some applications, the arms elastically or otherwise automatically contract on the tissue, in order to cause closure of the passage. Alternatively or additionally, a suture or wire is coupled to each flap, and the physician pulls the suture or wire to cause the arms to clamp onto the tissue. Further alternatively or additionally, the arms are plastically deformed, when brought together, and a tool is used for plastically deforming the arms. For some applications, the clip has a high friction surface facing the heart, e.g., on the base portion and/or the arms of the clip, which inhibits motion of the clip with respect to the heart surface.

For some applications, an extracardiac and/or an intracardiac fixation device is operated in combination with the clip, in order to maintain the clip in place. For example, an intracardiac surface may be coupled to the clip, the intracardiac surface being larger than the hole in the apex after the hole has been closed, and thereby preventing the clip from separating from the external surface of the heart. Alternatively or additionally, the clip is stabilized by a stabilizing element, e.g., by being pressed by a balloon coupled that is between the heart and the chest wall (or another layer), and/or by a balloon or other securing mechanism that is outside of the skin and coupled to the clip by a tether. FIG. 7 shows suitable configurations for balloons that may be used to stabilize the clip. For some applications, the tube coupling the stabilizing element to the clip additionally serves as a drainage tube, carrying fluid from a hole in the clip away from the heart, and releasing the fluid for example through holes in the side of the drainage tube.

For some applications, the clip is advanced to the heart prior to creating the passage in the heart wall. The clip is typically placed on the external side of the heart, such that the clip surrounds the area in which the passage will be made, in accordance with the procedures described herein. The clip is typically maintained in an uncompressed, open configuration thereof by any suitable mechanism or device, e.g., a rigid cannula that passes through a lumen in the clip and contains a hemostatic valve. While performing the procedures in which the passage in the heart wall is made and/or subsequently thereto (e.g., during implantation of a prosthetic valve), the clip typically reduces leakage of blood around the cannula. Alternatively, the clip is placed at the apex following the main procedure (e.g., implantation of a prosthetic valve).

Reference is now made to FIGS. 11A-D, which are schematic illustrations of a suturing system 100 for sealing a passage in the heart, in accordance with some applications of the present invention. For some applications, additional systems and methods for percutaneously sealing a passage in a hollow organ (such as the heart) are provided. For example, the passage in the heart wall may be sealed by suturing of the passage, as described with reference to FIGS. 11A-D, in accordance with some applications of the present invention. As provided by some applications of the present invention, a guidewire 10 and/or 15 is placed between the heart and the skin of a subject. Subsequently, a path is created between the heart and the skin of a subject by passing cannula 60 over the guidewire towards the heart. Cannula 60 has distal and proximal ends thereof, and the distal end of outer tube 64 of the cannula is placed in contact with an external side of the heart wall, over the guidewire. For some applications, suction cup 77 is disposed at the distal end of the outer tube of the cannula, which facilitates contact of the cannula with the external side of the heart wall. Typically, suction is applied via the suction cup, to create a seal surrounding the area of the passage on the external side of the heart, in order to reduce leakage of blood by the beating heart into the thorax, during performance of the procedures described herein. Subsequently, inner tube 62 of cannula 60 is advanced through outer tube 64 towards the heart chamber, e.g., left ventricle 4. Inner tube 62 typically has distal and proximal ends thereof, and the distal end is configured to penetrate the passage in the wall of the heart.

For some applications, at least one long surgical needle 102 with a suture is advanced through inner tube 62 towards the heart as shown in FIG. 11A. The long needle typically comprises a curved distal end, e.g., a distal end that is shaped to define a “J” configuration. For some applications, a portion of surgical needle 102 is more flexible in one region thereof (e.g., at the curve of the J) and less flexible at another region thereof (e.g., along the straight portion of the J). Alternatively, the stiffness of the needle is the same throughout and, for example, the needle is pre-shaped and/or otherwise configured such that it springs open to the curved configuration described with reference to FIG. 11A as the needle exits the sheath (e.g., the inner tube) which constrains its shape. The needle is advanced out of the inner tube, into the heart chamber, and pulled towards the heart tissue, such that the curved distal end of the needle is passed through the tissue, e.g. into the space between inner tube 62 and suction cup 77, as shown in FIG. 11B. For some applications, as described, a suture 104 (shown in FIG. 11C) is pre-attached to the needle for suturing the passage. (Alternatively, a suture is advanced through the space between inner tube 62 and suction cup 77 towards the curved distal end of the needle. This suture is then coupled to the needle, and the needle with the suture is retracted back through the heart tissue, in order to facilitate subsequent closure of the passage.)

The suture typically comprises proximal, distal, and tissue-engaging portions. The proximal portion typically remains outside of the body of the subject. As shown in FIG. 11C, the distal portion of the suture is passed through the cardiac tissue, and through the space between inner tube 62 of cannula 60 and suction cup 77 (suction cup 77 typically comprising a flared distal end of outer tube 64). The suture is typically then passed through outer tube 64, toward the skin of the subject. The distal end may be pulled through outer tube 64 of the cannula by an additional tool, e.g., forceps. Eventually, both the proximal and distal portions of the suture are positioned outside the body of the subject and may be manipulated by the surgeon, e.g., the surgeon may bring both portions together into a knot. Typically, a single closure suture stitch is obtained across the passage in the heart wall. The suturing procedure may be repeated until sufficient sealing of the passage is achieved. For some applications, a pair of sutures are passed through the cardiac tissue as described (e.g., (1) at 12 and 6 o'clock, and/or (2) at 3 and 9 o'clock, with respect to the inner tube), and the pair of sutures are tied to each other, in order to close the passage in the heart. FIG. 11D shows pairs of suture stitches made in accordance with applications of the present invention, in order to seal the passage in the heart wall.

It is to be noted that any suturing procedure described herein may be applied to seal any passage made in a wall of a body lumen, by placing a guidewire between the body lumen and skin of a subject and advancing the suture through the inner and outer tubes as described.

For some applications, needle 102 comprises a hollow needle shaped to define a needle lumen. Typically, the curved distal end which is shaped to define a “J” configuration is 8-150 mm (e.g., 15-100 mm) in length, measured along the length of the needle, from the point where the curving begins until the distal tip of the needle. (It is to be understood that lengths shorter or longer than these are appropriate for various applications.) In addition, at the time when the distal tip of the needle is about to penetrate tissue, the tip is typically oriented in a proximal direction and is substantially parallel to the straight portion of the “J” configuration.

For some applications, while being passed into the body toward the heart, the distal tip of needle 102 is aimed in a distal direction, i.e., toward the heart. Alternatively, the distal tip is aimed in a proximal direction, i.e., away from the heart, during passage of the needle toward the heart. In this case, the needle in effect has a relatively sharp bend in order to allow it to be passed into the body through inner tube 62. In order to provide this relatively sharp bend, needle 102 may, for example, comprise nitinol and may have one or more slits at the desired site for the relatively sharp bend, which facilitate the bending of needle 102. For some applications, once needle 102 is within the heart chamber and is no longer constrained by inner tube 62, the desired curvature of the needle for suturing is automatically attained, due to shape-memory attributes of the needle. Alternatively or additionally, a control wire coupled near the distal tip of needle 102 is used to apply a force to the needle to control the extent of curvature of the needle.

The needle is advanced out of the inner tube, into the heart chamber, until the entire curved distal end is positioned in the heart chamber. Subsequently, the needle is pulled towards the heart tissue (typically without rotating the needle along a path of insertion of the needle, as is common with surgical suturing), such that the curved distal end of the needle is passed through the tissue into the space between suction cup 77 and inner tube 62.

In addition, for some applications, a guidewire (not shown) is provided, having a proximal end and a distal end, and typically having a suture coupled to the proximal end thereof. The distal end of the guidewire is advanced through the lumen of the needle, through the left ventricle and the tissue, and into the space between suction cup 77 and inner tube 62, until it emerges from the proximal end of outer tube 64. The guidewire is thus advanced such that the suture coupled to the guidewire is pulled, by the guidewire, through the lumen of the needle, through the left ventricle and the tissue and into the space between outer tube 64 and inner tube 62. Typically, the suture is advanced through the tissue in a direction that is parallel to the axis of the passage in the heart. The guidewire and suture are then pulled through outer tube 64 towards skin of the subject. Eventually, both the proximal and distal portions of the suture are positioned outside the body of the subject and may be manipulated by the surgeon, e.g., the surgeon may bring both portions together into a knot and advance the knot towards the passage in the heart for sealing of the passage. The suturing procedure may be repeated until sufficient sealing of the passage is achieved.

For some applications, a flexible or substantially rigid cardiac patch (not shown) is attached to the sutures, e.g., by passing the sutures through holes in the patch, or by the cardiac patch being packaged in a kit, pre-attached to the sutures. For cases in which the patch is generally rigid, it may be shaped generally like a shirt button. In either case, the patch is typically advanced along the sutures, through inner tube 62, until the patch is situated in the heart chamber, such that the patch covers the passage in the heart tissue and is secured in place by the sutures. The sutures may be further pulled in order to pull the patch against the inner surface of the heart chamber to seal the passage in the heart wall. Alternatively, the patch may be positioned outside of the heart chamber. For applications in which the patch is within the heart, as well as for applications in which the patch is outside of the heart, even if part of the patch is rigid, the portion of the patch that contacts the heart is typically at least somewhat compliant.

Reference is again made to FIG. 11A. For some applications, a length D2 of a post-curve distal region of needle 102 is 3-200 mm, such as 5-150 mm (e.g., 10-20 mm). The distance D1 between the longitudinal elongated portion of the needle and the distal sharp tip is 2-15 mm (e.g., 4-8 mm). A length D3 of the curved portion of the needle is 3-22 mm. For some applications, the smallest radius of curvature along the J-shaped needle is 1-8 mm. Typically, needle 102 is pulled back proximally from within the heart chamber, such that the needle penetrates the layers of the heart, from within the chamber until it passes out of the heart and out of the pericardium, into the space surrounding the pericardium, as shown.

For some applications, needle 102 is able to assume its J configuration and to return to its straight configuration either without any special preparation of the J needle to allow this, or, alternatively, using a method such as slotting the tube from which the needle is made at the area of the curvature of the J needle, and bending the edge with a wire coupled to the distal end of the needle running along the entire length of the needle.

For some applications, techniques are practiced in accordance with those described with reference to FIGS. 14C(I)-14C(VIII) of U.S. Provisional Patent Application 61/452,465, and/or with reference to FIGS. 14C(I)-14C(VIII) of U.S. Provisional Patent Application 61/475,751, both of which applications are incorporated herein by reference, mutatis mutandis. For some applications, pledgets (not shown) are advanced over sutures 104 to the internal and external surfaces of the heart using pushers (not shown). Pledgets are useful in some cases for suture tear-out prevention when applying force for closing the hole at the apex. For some applications, flexion elements are used to facilitate the flexing of the pushers, according to the suture trajectory. The pledgets can assume a simple rectangular shape or an accordion-like geometry, for reinforcing the suture tear out resistance and/or for providing more surface coverage. The pledgets typically comprise PTFE, ePTFE, PET or any other suitable plastic material and/or fabric and/or biodegradable and/or bioabsorbable polymer.

For some applications, a pledget and/or a patch (e.g., serving as a bandage) is advanced and placed in contact with the internal or external surface of the heart. For example, a folded pledget or bandage or patch may be advanced along two or more sutures to form a patch at the inner surface of the heart (not shown). For some applications, a cylindrical tube pusher is used to advance the patch to the inner surface of the heart.

For some applications, a button-like sealing element is parachuted along two or more of sutures 104, using a pusher. The sealing element typically comprises a soft (e.g., sponge-like) portion that faces the surface of the heart and typically adapts itself to the specific geometry of the heart and allows applying compression against the heart without causing trauma, while keeping contact with the curved apex around the hole throughout the cardiac cycle. A second portion of the sealing element is generally rigid, such that tying a knot, and thereby applying force by the suture with respect to the rigid portion of the sealing element, inhibits transverse shear motion of the suture loop, thereby inhibiting the suture from cutting through the myocardium. For some applications, the sealing element comprises PTFE, ePTFE, PET or any other suitable plastic material and/or fabric and/or biodegradable and/or bioabsorbable polymer.

For some applications, the techniques for closing a hole in a subject's heart described with reference to FIGS. 11A-D are practiced in combination with those described with reference to FIGS. 9A-10D.

The procedures described hereinabove are described with reference to the left ventricle of the heart by way of illustration and not limitation. It is to be noted that any of the above mentioned procedures may be performed on any heart chamber as appropriate. For applications in which access to the left side of the heart is desirable, percutaneous cardiac catheterization through the femoral or radial artery is performed. Typically, applications that provide access to the left ventricle are particularly suitable for cardiac procedures such as aortic valve and/or mitral valve repair and/or replacement. It is to be noted that any other percutaneous cardiac catheterization procedure known in the art can be used to gain access to the left side of the heart, e.g., via the femoral vein and through a foramen ovale in the wall between the atria. For some applications, femoral vein catheterization in a retrograde direction is performed, in order to gain access to the right side of the heart. Access to the right side of the heart is particularly suitable for cardiac procedures such as, by way of illustration and not limitation, pulmonary valve and/or tricuspid valve repair or replacement.

Furthermore, it is noted that although techniques are described hereinabove by way of example, the scope of the present invention includes performing similar techniques on other organs or lumen, such as other sites in the cardiovascular system, the stomach, or the urinary bladder.

It is to be noted that any of the procedures described herein may be conducted under fluoroscopy or any other image guidance known in the art.

It is additionally noted that although some embodiments of the present invention are described hereinabove with respect to use of a catheter passed into the femoral artery (or another peripheral blood vessel) over a guidewire, the scope of the present invention includes passing a single guidewire into the peripheral vessel, into a chamber of the heart, and subsequently creating a passage in the wall of the heart using the guidewire and passing the guidewire through the passage, until the guidewire reaches the skin. The distal tip of the guidewire may be used to puncture or electrically ablate the wall of the heart, in order to create the passage.

For some applications, some or all of the components usable in a given procedure described hereinabove are packaged in a kit.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-13. (canceled)

14. Apparatus for use with an insertion device, the apparatus comprising a closure device, the closure device comprising:
a plug portion configured to be placed within a passage in a wall of a heart of a subject by being introduced to the passage via the insertion device, the insertion device being configured to maintain the plug portion in a constrained state thereof during the insertion, the plug portion being configured to automatically increase a radius of the plug portion by more than 0.5 percent by assuming a non-constrained state thereof by being pushed out of a distal end of the insertion device;
an intracardiac portion, coupled to the plug portion, and configured for placement within a heart chamber; and
an extracardiac portion coupled to the plug portion and configured for placement outside of the heart chamber.

15. The apparatus according to claim 14, wherein the plug portion comprises a soft outer layer thereof.

16. The apparatus according to claim 14, wherein a radius of the passage is defined by an outer radius of the insertion device, wherein while the plug portion is in the constrained state thereof, the radius of the plug portion is less than the outer radius of the insertion device, and wherein upon assuming the non-constrained state thereof, the radius of the plug portion is at least equal to the outer radius of the insertion device.

17. The apparatus according to claim 14, wherein upon assuming the non-constrained state thereof, the plug portion is configured to seal the passage by expanding to occupy the passage.

18. The apparatus according to claim 14, wherein the plug portion is configured to automatically increase the radius of the plug portion by more than 5 percent upon assuming the non-constrained state thereof, by being pushed out of a distal end of the insertion device.

19. The apparatus according to claim 14, wherein the plug portion is configured to automatically increase the radius of the plug portion by less than 100 percent upon assuming the non-constrained state thereof, by being pushed out of a distal end of the insertion device.

20. The apparatus according to claim 14, wherein the plug portion is configured to increase the radius of the plug portion even in an absence of any radial expansion of the plug portion that is due to absorbance of fluid by the plug portion.

21-22. (canceled)

23. The apparatus according to claim 14, wherein the plug portion is configured, in the absence of any force applied to the plug portion, to reduce a length of the plug portion by 0.5-50 percent.

24-27. (canceled)

28. The apparatus according to claim 14, wherein the intracardiac portion is shaped to define a disc that is concave in a distal direction.

29. The apparatus according to claim 28, wherein the extracardiac portion is shaped to define a disc that is concave in the distal direction.

30. The apparatus according to claim 29, wherein a radius of curvature of the extracardiac portion is less than a radius of curvature of the intracardiac portion.

31. The apparatus according to claim 14, wherein the intracardiac portion, the extracardiac portion and the plug portion are moveable with respect to each other.

32. The apparatus according to claim 31, wherein the closure device is configured to conform with anatomical variations of the subject's heart, by the intracardiac portion, the extracardiac portion and the plug portion being moveable with respect to each other.

33. The apparatus according to claim 31, wherein the closure device is configured to seal the passage in the wall of the heart by being placed inside the passage, and wherein the closure device is configured to maintain the seal of the passage, by the intracardiac portion, the extracardiac portion and the plug portion being moveable with respect to each other.

34. The apparatus according to claim 14, wherein the intracardiac portion and the extracardiac portion of the closure device are configured to be maintained in folded configurations thereof during insertion of the closure device via the insertion device, and wherein the intracardiac portion and the extracardiac portion are configured to automatically assume unfolded states thereof by being pushed out of the distal end of the insertion device.

35. The apparatus according to claim 34, wherein the intracardiac portion and the extracardiac portion comprise a shape memory material that is configured to cause the intracardiac portion and the extracardiac portion to automatically assume the unfolded states.

36. The apparatus according to claim 14, wherein the plug portion is configured to increase the radius of the plug portion upon absorbing body fluid.

37. The apparatus according to claim 36, wherein the plug portion is configured to reduce the length of the plug portion by 0.5-50 percent, upon absorbing the body fluid.

38-42. (canceled)

43. Apparatus comprising a closure device, the closure device comprising:
a plug portion configured for placement within a passage in a wall of a heart of a subject;
an intracardiac portion, coupled to the plug portion, and configured for placement within a heart chamber, and having a radius of curvature; and
an extracardiac portion coupled to the plug portion and configured for placement outside of the heart chamber, having a radius of curvature that is less than the radius of curvature of the intracardiac portion.

44-111. (canceled)

112. A method comprising:
passing a closure device over a guidewire toward a passage in a wall of a heart of a subject, the closure device including:
a plug portion configured for placement within the passage;
an intracardiac portion, coupled to the plug portion, and configured for placement within the heart chamber;
an extracardiac portion coupled to the plug portion and configured for placement outside the heart chamber; and
sealing the passage, by inserting the closure device through the passage such that the plug portion is maintained in place within the passage.

113. The method according to claim 112, wherein the plug portion includes an intramural plug portion and inserting the closure device through the passage comprises passing the intramural plug portion through the passage.

114. The method according to claim 113, wherein inserting the closure device through the passage comprises introducing the insertion device to the passage via an insertion device, the insertion device being configured to maintain the plug portion in a constrained state thereof during the insertion, the plug portion being configured to automatically increase a radius of the plug portion by more than 0.5 percent by assuming a non-constrained state thereof by being pushed out of a distal end of the insertion device

115-117. (canceled)

118. The apparatus according to claim 14, wherein the plug portion does not include any rigid materials therein.
